SEQUENCE LISTING (4 sequences)

SEQ ID NO: 1 - protein, 105 aa, Artificial Sequence
  synthetic peptide
VMTQSPKFMSTSVGDRISVTCKASQDVGPNVAWYQQKPGQSPKPLIYSASYLYNGVPDRFTGSGSGTDFSLTISNVQSDDLAEYFCQQYNTYPFTFGGGTKLEIK

SEQ ID NO: 2 - protein, 123 aa, Artificial Sequence
  synthetic peptide
EVQLEESGGGLVQPKGSLKLSCAASGFTFNTYAMNWVRQAPGKGLEWIVRIRSKSNNYATYYVDSVKDRFTISRDDSQSMLYLQMNNLKTEDTAMYYCVTSYYDYDKVLFAYWGQGTTVTVKG

SEQ ID NO: 3 - protein, 387 aa, Artificial Sequence
  synthetic polypeptide
MDFQVQIFSFLLISASVIMSRGDIVMTQSPKFMSTSVGDRISVTCKASQDVGPNVAWYQQKPGQSPKPLIYSASYLYYGVPDRFTGSGSGTDFSLTISNVQSDDLAEYFCQQYNTYPFTFGGGTKLEIKGSTSGSGKSSEGKGEVQLEESGGGLVQPKGSLKLSCAASGFTFNTYAMNWVRQAPGKGLEWIVRIRSKSNNYATYYVDSVKDRFTISRDDSQSMLYLQMNNLKTEDTAMYYCVTSYYDYDKVLFAYWGQGTTVTVKGKHLCPSPLFPGPSKPFWVLVVVGGVLACYSLLVTVAFIIFWVRSKRSRLLHSDYMNMTPRRPGPTRKHYQPYAPPRDFAAYRSQVRKAAITSYEKSDGVYTGLSTRNQETYETLKHEKPPQ

SEQ ID NO: 4 - DNA, 1177 bp, Artificial Sequence
  synthetic polynucleotide
atggattttcaggtgcagattttcagcttcctgctaatcagtgcctcagtcataatgtctagaggagatattgtgatgacccagtctccaaaattcatgtccacatcagtaggagacaggatcagcgtcacctgcaaggccagtcaagatgtgggtcctaatgtagcctggtatcaacagaaaccagggcaatctcctaaaccactgatttactcggcatcctacctatattatggagtccctgatcgcttcacaggcagtggatctgggacagatttctctctcaccatcagcaatgtgcagtctgatgacttggcagagtatttctgtcagcaatataacacctatccgttcacgttcggagggggcaccaagctggaaatcaaagggtcgacttccggtagcggcaaatcctctgaaggcaaaggtgaggtgcagctggaggagtctggtggaggattggtgcagcctaaagggtcattgaaactctcatgtgcagcctctggattcaccttcaatacctacgccatgaactgggtccgccaggctccaggaaagggtttggaatggattgttcgcataagaagtaaaagtaataattatgcaacatattatgtcgattcagtgaaagacaggttcaccatctccagagatgattcacaaagcatgctctatctgcaaatgaacaacttgaaaactgaggacacagccatgtattactgtgtgacttcttactatgattacgacaaggtcctgtttgcttactggggccaaggaactacggtcaccgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatagatctcaagtgcgaaaggcagctataaccagctatgagaaatcagatggtgtttacacgggcctgagcaccaggaaccaggagacttacgagactctgaagcatgagaaaccaccacagtagctttagactcgag

(19) United States
(12) Patent Application Publication
(10) Pub. No.: US 2022/0204928 A1
(43) Pub. Date: Jun. 30, 2022

(54) EXTRACELLULAR VESICLES DERIVED FROM ACTIVATED CAR-T CELLS

(71) Applicants: YEDA RESEARCH AND DEVELOPMENT CO. LTD., Reehovot (IL); ICHILOV TECH LTD., Tel Aviv (IL)

(72) Inventors: Anat AHARON, Kiryat Tivon (IL); Anat GLOBERSON LEVIN, Tel Aviv (IL); Irit AVIVI, Carmel (IL); Galit HORN, Hod Hashron (IL); Tova WAKS, Petach Tikva (IL); Zelig ESHHAR, Tel Yitzhak (IL)

(21) Appl. No.: 17/604,169

(22) PCT Filed: Apr. 16, 2020
(86) PCT No.: PCT/IL2020/050444

§ 371 Date: Oct. 15, 2021

(30) Foreign Application Priority Data
Apr. 18, 2019 (IL) 266153

(51) Int. Cl. C12N 5/0783; C07K 16/32; C07K 14/705; C07K 14/725; A61K 45/06; A61K 39/395; A61K 38/17; A61P 35/00; A61K 35/17; C07K 16/28

(52) U.S. Cl. CPC C12N 5/0636; A61K 2039/5156; C07K 14/70596; C07K 14/7051; A61K 45/06; A61K 39/39558; A61K 38/177; A61K 38/1774; A61P 35/00; A61K 35/17; C07K 16/2803; C07K 2319/30; C07K 2319/33; C07K 2317/76; C07K 16/32

(57) ABSTRACT

The present invention provides extracellular vesicles (EVs) derived from T-cells expressing chimeric antigen receptors (CAR) specifically activated with an antigen to which the CAR bind specifically, pharmaceutical compositions comprising these vesicles as well as their use in treating cancer. In particular the present invention exemplifies EVs derived from activated T-cells expressing CAR that bind specifically to HER2 cancer antigen, pharmaceutical composition comprising these EVs and their use in treating a cancer overexpressing HER2, such as ovarian cancer and breast cancer.

### FIELD OF THE INVENTION

The present invention relates to extracellular vesicles derived from activated T-cells expressing chimeric antigen receptors, pharmaceutical compositions comprising same and their use in treating cancer.

### BACKGROUND OF THE INVENTION

CAR T cells are engineered T cells expressing a chimeric antigen receptor (CAR) that recognizes a specific tumor associated antigen (TAA) which may distinguish cancer cells from healthy ones. CAR T cells are used as immunotherapy for several different oncologic diseases especially for leukemias and lymphomas in the past few years. Several methods are used to transduce or transfect T cell with CAR ex-vivo. These methods can include the use of viral vectors or other methods to introduce the DNA or RNA. As a result, the transfected T cell contains a genomic sequence for the specific protein and present or express the receptor. Upon recognition of the TAA, the CAR T cell is stimulated and can efficiently kill its target cells.

As a potent therapeutic modality, there are several adverse events that may be associated with the CAR T cell therapy, such as cytokine release syndrome (CRS) and life threatening cytokine storm. The side effects associated with CRS may include hypotension, hypoxia, high grade fever and neurological disturbances. Another significant challenge is overcoming the tumor microenvironment so that this treatment can be applied to treat solid tumors and not only hematologic malignancies.

Extracellular vesicles (EVs) are membrane vesicles secreted by different types of cells including blood cells. EVs can be divided into three subpopulations: (I) exosomes have a size of 30-100 nm in diameter and are derived from endosomal compartments; (II) microvesicles have a size of 100 nm-1 μm in diameter and are released from the cell surface via “vesiculation”; and (III) apoptotic bodies have a size of 1-5 μm in diameter and are released from apoptotic cells. EVs are present in the blood circulation under normal physiological conditions, and their levels are increased in a variety of diseases such as diabetes and related vascular complications, cardiovascular disease, hematologic malignancies as well as in solid tumors such as breast cancer. Tang et al., (Oncotarget 2015; 6(42): 44179-90) discussed in general terms different approaches for use of cellular and exosomal platforms for treatment of cancer. WO 2019/128952 describes method for preparing an immune cell exosomes carrying CAR obtained by isolation, and uses thereof.

EVs have been suggested to contain several elements of the parent cell from which they are derived, including proteins, DNA fragment, micro RNA and mRNA. Upon release, EVs can interact with target cells via a receptor mediated mechanism, or they can directly fuse with the plasma membrane of target cells, thus releasing their content into the recipient cell. Alternatively, EVs can be internalized via endocytosis and release their content into the cytosol of target cells.

There is an unmet need for development of additional approaches for safe and efficient treatment of cancer.

### SUMMARY OF THE INVENTION

The present invention is based on the observation that a population of isolated extracellular vesicles (EVs) which are derived from T-cells expressing a chimeric antigen receptor (CAR) following activation by exposure to antigen to which the CAR binds specifically, provided outstanding anti-cancer effects. It was found that a population with medium to large size EVs had improved apoptotic activity, whereas a population comprising mainly exosomes had weak to moderate activity.

Accordingly, provided herein are EV-based compositions and methods, providing improved anti-cancer therapy compared to known treatments. According to one aspect, the present invention provides isolated activated extracellular vesicles (EVs) derived from activated T-cells expressing a chimeric antigen receptor (CAR T-cells) wherein more than 25% of the EVs have a particle diameter size of more than 150 nm. In other words, the present invention provides isolated activated extracellular vesicles (EVs) derived from activated T-cells expressing a chimeric antigen receptor (CAR T-cells), wherein at least 25% of the EVs have a particle size of above 150 nm. The CAR T cells from which the EVs are derived/obtained are specifically activated CAR T cells, i.e. were activated with tumor associated antigen to which CAR binds specifically. Thus, the isolated EVs are denoted as activated EVs. According to some embodiments, at least 29% or at least 30% or at least 35% of the EVs have size above 150 nm. According to some embodiments, the mean size of the isolated activated EVs of the present invention is more than 140 nm. According to other embodiments, the mean size of the isolated activated EVs of the present invention is more than 150 nm. According to certain embodiments, the mean size of the isolated activated EVs of the present invention is more than 155 nm. According to certain embodiments, the mean size of the isolated activated EVs of the present invention is more than 160 nm. According to some embodiments, the EVs of the present invention present the CAR of the activated CAR T-cells from which the EVs are originated/derived.

According to the teaching of the present invention any CAR specific to tumor associated antigen may be used. According to some exemplary embodiments, the CAR is selected from anti-HER2, anti-CD19 and anti-CD38 CAR. According to more specific embodiments, the CAR is N29 CAR. As discussed below the isolate EVs of the present invention preserve the biological and therapeutically properties of the CAR T cells from which the EVs are derived. Thus, according to some embodiments, the have isolated EVs of the present invention EVs are cytotoxic EVs. According to some embodiments, the isolated EVs of the present invention express CD38, CD3 and/or HLARD antigens of their surfaces. According to some embodiments, more than 10%, 20 and/or 20% of the isolated EVs of the present invention express CD38, CD3 and/or HLARD antigens, respectively. According to some embodiments, the EVs of the present invention may further comprise an exogenously added anticancer agent.

According to another aspect, the present invention provides a pharmaceutical composition comprising a therapeutically effective amount of the isolated activated EVs of the present invention, and a pharmaceutically acceptable carrier. According to alternative embodiments, the pharmaceutical composition comprises the EVs of the present invention as a sole anti-cancer agent. According to some embodiments, the pharmaceutical composition further comprises an additional anti-cancer agent. According to one embodiment, the pharmaceutical composition of the present invention is for use in treating cancer. Non-limiting examples of cancer that may be treated using the EVs or the pharmaceutical composition of the present invention are ovarian cancer, breast cancer, lung adenocarcinoma, stomach, liver cancer, pancreatic cancer, brain cancers and a hematology malignancy. According to some embodiments, the cancer is a cancer presenting a tumor associated antigen to which the CAR of the CAR T-cell, from which the EVs of the present invention are derived, binds specifically. According to some embodiments, the pharmaceutical composition of the present invention is administered systemically or intra-tumorally.

According to another aspect, the present invention provides a method for treating cancer in a subject in need thereof comprising administering to the subject an effective amount of isolated activated EVs of the present invention, i.e. EVs derived from activated T-cells expressing a chimeric antigen receptor (CAR T-cells). According to yet another aspect, the present invention provides a method for preparation of the isolated activated extracellular vesicles of the present invention, said EVs are derived from activated CAR T-cells, wherein the method comprises incubating CAR T-cells with a tumor associated antigen to which the CAR binds specifically under conditions enabling T cell stimulation, and isolating the derived activated extracellular vesicles. According to more specific embodiments, the method of preparation of the EVs of the present invention comprises: (1) incubating CAR T-cells with a tumor associated antigen to which the CAR binds specifically in cell medium under conditions enabling T cell activation; (2) separating the CAR T-cells from cell medium comprising the EVs; and (3) isolating the derived activated extracellular vesicles, thereby obtaining isolated activated EVs of the present invention, wherein at least 22% or at least 25% of the EVs have size of 150 nm or more. According to some embodiments, isolation of the EVs comprises centrifugation at centrifugation force of at from 8,000×g to 30,000×g for from 0.5 to 4 hours. Thus, according to some embodiments, the method of preparation of the EVs of the present invention comprises: (1) incubating CAR T-cells with a tumor associated antigen to which the CAR binds specifically in cell medium under conditions enabling T cell activation; (2) separating the CAR T-cells from cell medium; and (3) isolating the derived activated extracellular vesicles by centrifuging at from 8,000 to 25,000×μ or from 8,000 to 12,000×g for from 30 to 210 min, thereby obtaining isolated activated EVs of the present invention, wherein at least 25% or at least 25% of the EVs have size of 150 nm or more. According to other embodiments, the present invention provides isolated activated extracellular vesicles prepared by the preparation methods of the present invention. In some specific embodiments, the present invention provides isolated activated extracellular vesicles prepared by a method comprising (1) incubating CAR T-cells with a tumor associated antigen to which the CAR binds specifically in cell medium under conditions enabling T cell activation; (2) separating the CAR T-cells from cell medium; and (3) isolating the derived activated extracellular vesicles by centrifuging at from 8,000 to 25,000×g or from 8,000 to 12,000×g for from 30 to 210 min. The resulted preparation of the EVs comprises at least 25% of EVs having size above 150 nm. According to some embodiments, the mean size of the isolated EVs is about 140 nm or more. According to some aspects, the present invention provides extracellular vesicles prepared and isolated according to method of preparation of the present invention. Thus, in some embodiments, the invention provides extracellular vesicles obtainable by the method of preparation of the present invention. The full scope of the invention will be better understood together with the figures, description, examples and claims that follow.

### BRIEF DESCRIPTION OF DRAWINGS

FIG. 1 shows size distribution (diameter in nm) measured by Nanoparticle-tracking analysis (NTA) of EVs obtained from N29 CAR T-cells (FIG. 1A) and un-transduced (UT) T-cells (FIG. 1B), both incubated with HER2 presenting cancer cells. FIG. 1C-1E summarize data of 5-9 experiments on EVs pellet obtained by 5 different centrifugation protocols as described in the method section. Briefly: Method 1—Centrifuged for 60 minutes at 20,000×g. Method 2—The supernatant obtained in Method 1 was further centrifuged for 60 min at 100,000×g; Method 3—centrifuged for 30 min at 10,000×g. Method 4—supernatant of Method 3 was further centrifuged for 110 min at 70,000×g. Method 5—Centrifuged for 180 min at 10,000×g; Sample 1—N29 on SKOV; Sample 2—N29 on OVCAR; Sample 3—UT on SKOV; Sample 4—UT on OVCAR; Sample 5—N29 on medium; Sample 6—UT on medium; Sample 7—SKOV on medium; and Sample 8—OVCAR on medium.

FIG. 1C summarizes the EVs size for all Samples and Methods; FIG. 1D shows statistics for Samples 1 and 2 isolated by Methods 1-5.

FIG. 1E shows the percentage of large EVs (>150 nm diameter) for all Samples and Methods.

FIG. 1F shows statistics for Samples 1 and 2 isolated by Methods 1-5.

FIG. 1G shows the number of EVs having size above 150 nm in preparations obtained by Method 1 and Method 5.

FIGS. 1H-J show side scatter (SSC) versus forward scatter (FSC) plots using 0.75 μm beads (FIG. 1H), plots of EVs from non-transduced T-cells (FIG. 1I) or EVs from T-cells transduced with N29 CAR (FIG. 1J).

FIG. 2 shows expression of markers on EVs isolated by Method 1: FIG. 2A-D show labeling (percentage) of unstained EVs obtained from: non-transduced non-incubated (2A) T-cells transduced with N29 and not activated (2B) N29 CAR T-cell not activated (2C) and N29 CAR T-cell stimulated with SKOV (2D). FIG. 2E-H shows labeling with isotype control IgG APC (percentage) of EVs obtained from: non-transduced non-stimulated (2E) T-cells transduced with N29 and not activated (2F) N29 CAR T-cell not stimulated (2G) and N29 CAR T-cell stimulated with SKOV (2H). FIG. 2I-L shows labeling with Anti CD3-APC (percentage) of EVs obtained from non-transduced non-stimulated (2I) T-cells transduced with N29 and not activated (2J) N29 CAR T-cell not stimulated (2K) and N29 CAR T-cell stimulated with SKOV (2L). Anti-CD3 antibodies bind to the T cell receptor (TCR) complex on a mature T lymphocyte.

FIG. 2M summarizes membrane antigen expression of CD3, CD38 and HLADR on EVs pellet obtained from by Method 1 (20,000 g, 60 min) and Method 2 (100,000 g, 60 min) (n=5-9 experiments).

FIG. 3 shows labeling (percentage) of EVs obtained by Method 1 from unstained non-transduced T-cells (3A), unstained transduced with N29-GFP CAR (3B) or from non-transduced T-cells stained with isotype control IgG (3C).

FIG. 4 shows apoptotic effect of 6 hours exposure of HER2+SKOV cells (FIG. 4A) or HER2-OVCAR cell (FIG. 4B) to different samples of EVs obtained by Method 1. EVs obtained from: 1—Sample 1 (T cells expressing N29 CART after stimulation with SKOV) 25 μg/cell; 2-Sample 1, 12.5 μg/cells; 3—Sample 2 (T cells expressing N29 CAR T after incubated with OVCAR) 12.5 μg/cells; 4—Sample 4 (Non-transduced T cells incubated with SKOV) 25 μg/cell; 5—Sample 4 (Non-transduced T cells incubated with OVCAR) 25 μg/cell.

FIG. 4C shows effect of fresh or thawed EVs from Samples 1 or 2 each obtained by Method 1 or Method 2 (detected by flourcent microscopy, analysed by Imaj J saftwar); FIG. 4D summarize the effects or EVs on SKOV Her2+ cells and OVCAR Her2-cells after 6 h of exposure;

FIG. 4E summarizes the apoptotic effect of the 8 different EVs populations (in two different concentrations: 50 μg and 25 μg EVs) obtained by Method 1 (dark gray columns) and Method 2 (light gray columns).

FIG. 5 shows microscopy images of SKOV cells incubated for 20 hours with EVs obtained from different CAR-T cells as following: FIG. 5A—EVs from N29 CAR-T pre-stimulated with SKOV cells; FIG. 5B—EVs from anti-CD19 CAR-T pre-stimulated with SKOV cells; FIG. 5C EVs from N29 CAR-T pre-stimulated with Raji cells; and FIG. 5D—EVs from anti-CD19 CAR-T pre-activated with Raji cells.

FIG. 6 shows images of SKOV cells incubated for 40 hours with EVs obtained from different CAR-T cells as following: FIG. 6A—two images: EVs from N29 CAR-T pre-stimulated with SKOV cells; FIG. 6B—EVs from anti-CD19 CAR-T activated with SKOV cells; FIG. 6C—shows effect of EVs obtained from N29 CAR T stimulated with HER2+ cells on breast cancer HER2 negative cells.

FIG. 7 shows phase images and labeled cells with fluorescent Caspase 3/7 activity dye and cytotoxic dye (both used for real-time quantification of cell death). Images documented in white spots of SKOV cells treated with EVs from Sample 1 or 2 isolated by Methods 1, 3 and 5 (FIG. 7A) or by Methods 2 or 4, or treated with staurosporine (FIG. 7B). Imaging of SKOV cells was done by Incucyte.

FIG. 8 show magnified microscopic images of SKOV cells after 4 days exposure to 2 different concentrations of EVs of Sample 1 or EVs of Sample 2 isolated by 5 Methods. FIGS. 8A-8E: Sample 1, 50 μg, Methods 1-5, respectively; FIGS. 8F-8J: Sample 1, 25 μg, Methods 1-5, respectively; FIGS. 8K-80: Sample 2, 50 μg, Methods 1-5, respectively; FIGS. 8P-8T: Sample 2, 25 μg, Methods 1-5, respectively; and FIG. 8U—treatment with staurosporine.

FIG. 9 show magnified microscopic images of OVCAR cells after 4 days exposure to 2 different concentrations of EVs of Sample 1 or EVs of Sample 2 isolated by 5 Methods. FIGS. 8A-8E: Sample 1, 50 μg, Methods 1-5, respectively; FIGS. 8F-8J: Sample 1, 25 μg, Methods 1-5, respectively; FIGS. 8K-80: Sample 2, 50 μg, Methods 1-5, respectively; FIGS. 8P-8T: Sample 2, 25 μg, Methods 1-5, respectively; and FIG. 8U—treatment with staurosporine.

FIG. 10 shows kinetics of incorporation of fluorescent Caspase 3/7 activity marker in SKOV cells after exposure to EVs of Samples 1 and 2 obtained by Methods 1, 3 and 5. The treatments are enumerated as: 1—Method 1, Sample 1, 50 μs; 2—Method 1, Sample 1, 25 μg; 3—Method 3, Sample 1, 50 μg; 4—Method 3, Sample 1, 25 μg; 5—Method 5, Sample 1, 50 μg; 6—Method 5, Sample 1, 25 μg; 7—Method 1, Sample 2, 50 μg; 8—Method 1, Sample 2, 25 μg; 9—Method 3, Sample 2, 50 μg; 10—Method 3, Sample 2, 25 μg; 11—Method 5, Sample 2, 50 μg; 12-Method 5, Sample 2, 25 μg.

FIG. 11 shows kinetics of incorporation of fluorescent Caspase 3/7 activity marker in SKOV cells after exposure to different EVs as of Samples 1 and 2 obtained by Methods 2 or 4, or after exposure to Staurosporine. The treatments are enumerated as: 1—Method 2, Sample 1, 50 μg; 2—Method 2, Sample 1, 25 μg; 3—Method 4, Sample 1, 50 μg; 4—Method 4, Sample 1, 25 μg; 5—Staurosporine 1:100; 6—Method 2, Sample 2, 50 μg; 7—Method 2, Sample 2, 25 μg; 8—Method 4, Sample 2, 50 μg; 9—Method 4, Sample 2, 25 μg.

FIG. 12 summarized the total caspase activity of EVs of Sample 1 and 2 (2 concentrations) isolated by Methods 1-5 after 4^{th} day of incubation.

FIG. 13 shows the viability/proliferation of SKOV cells incubated for 40 hours with EVs derived from Sample 1 or from Sample 3, purified by Method 1 as measured after 48 hours by XTT method.

FIG. 14 shows cytotoxic effect of activated EVs on MDA231 HER2+ cells. EVs obtained from N29 on MDA231 HER2+ or from non-transduced T cells on MDA 231-HER2+ cells. MDA231 HER2+ cells were exposed to EVs in 3 different dilutions, and were measured after 48 hours by CytoTox 96, Cytotoxicity Assay.

### DETAILED DESCRIPTION OF THE INVENTION

The present invention relates to a population of extracellular vesicles (EVs), wherein the EVs are derived from activated T-cells expressing a chimeric antigen receptor (CAR), wherein at least 25% of the EVs have size of 150 nm or more.

It was surprisingly found that preparation of EVs that were derived from T-cells expressing N29 CAR specific to HER2, (denoted as N29 CAR T) and activated with ovarian cancer cells presenting HER2, and wherein the population of the EVs comprises more than 25% of EVs having size above 150 nm, exhibited an outstanding cytotoxic effect towards ovarian and breast HER2 positive cancer cells. It was further shown that the effect was not lost upon freezing the EVs. Thus, the EVs may be frozen and thawed before use, which ease their handling. This superior cytotoxic effect was not observed for EVs derived from CAR T-cells that were not pre-stimulated or incubated with non-related antigens (such as CD19, related to hematology cancer or cancer cells which are HER2 negative). Moreover, preparation of EVs from specifically activated N29 CAR T cells comprising 20% or less EVs having size of above 150 had much weaker effect. It was further found that EVs obtained from the pre-stimulated CAR T cells were distinguishable from EVs of control preparations by their physical properties such as size distribution and presence of certain antigen markers. It is understood that EVs derived from stimulated N29 CAR T-cells having the specific properties as described in this application is merely a proof of concept. EVs from T-cells comprising CARs specific to other tumor associated antigens or EVs obtained from T-cell comprising a plurality of such CARs may be used according to the teaching of the present invention, as long as the EVs have the properties as described in the present application and in particular the size as described.

According to one aspect, the present invention provides isolated activated extracellular vesicles (EVs) derived from activated T-cells expressing a chimeric antigen receptor (CAR T-cells), wherein at least 22% of the EVs have particle diameter size of above 150 nm. According to some embodiments, the present invention provides isolated activated extracellular vesicles (EVs) derived from activated T-cells expressing a chimeric antigen receptor (CAR T-cells), wherein at least 22% of the EVs have size of above 150 nm. According to other embodiments, the present invention provides isolated activated extracellular vesicles (EVs) derived from activated T-cells expressing a chimeric antigen receptor (CAR T-cells), wherein at least 25% of the EVs have size of above 150 nm. In particular, embodiments of the invention is directed to isolated activated EVs derived from CAR T-cells activated by a CAR-mediated stimulation prior to EV isolation.

The terms “extracellular vesicles” and “EVs” are used herein interchangeably and refer to a cell-derived vesicles comprising a membrane that encloses an internal space. Generally extracellular vesicles range in diameter from 30 nm to 1000 nm, and may comprise various cargo molecules either within the internal space, displayed on the external surface of the extracellular vesicle, and/or spanning the membrane. Said cargo molecules may comprise nucleic acids, proteins, carbohydrates, lipids, small molecules, and/or combinations thereof. The term extracellular vesicles comprises also the terms “exosome” and “microvesicles”. The terms “exosomes” and “nanovesicle” are used herein interchangeably and refer to EVs having the size of between 30 to 100 nm in diameter. The term “microvesicles” as used herein refer to EVs having the size of between 100 to 1000 nm in diameter. Generally, the EVs may comprise at least a part of the molecular contents of the cells from which they are originated, e.g. lipids, fatty acids, polypeptides, polynucleotides, proteins and/or saccharides. According to the teaching of the present invention at least 25% of the EVs have size of above 150 nm. Alternatively, at least 25% of the EVs have size of 150 nm or more.

The extracellular vesicles of the present invention are mostly spherical and the terms “size”, “particle size”, and “particle diameter size” used herein interchangeably refer to the diameter of the extracellular vesicles or to the longer diameter of the extracellular vesicles. Any known method for measurement of particle size may be used to determine the size of the EVs of the present invention. A non-limiting example is nanoparticle-tracking analysis (NTA).

According to another embodiment, the isolated activated EV are microvesicles. According to a further embodiment, the isolated activated EVs are a combination of small and large vesicles.

According to some embodiments, at least 10% or at least 15% of the isolated activated EV have a size between 150 to 300 nm. According to some embodiments, at least 27% of the activated EVs have a particle size of 150 nm or more. According to one embodiment, at least 28% of the activated EVs have a size of 150 nm or more. According to one embodiment, at least 28% of the activated EVs have a size of more than 150 nm. According to one embodiment, at least 29% of the activated EVs have a size of 150 nm or more. According to one embodiment, at least 30% of the activated EVs have a size of 150 nm or more. According to one embodiment, at least 32% of the activated EVs have a size of 150 nm or more. According to one embodiment, at least 35% of the activated EVs have a size of 150 nm or more. According to another embodiment, at least 40% of the activated EVs have a size of above 150 nm. According to another embodiment, at least 42% of the activated EVs have a size of above 150 nm. According to yet another embodiment, at least 45% of the activated EVs have a size of above 150 nm. According to another embodiment, at least 50% of the activated EVs have a size of above 150 nm. According to another embodiment, at least 55% of the activated EVs have a size of above 150 nm. According to some embodiments, at least 60% of the activated EVs have a size of above 150 nm. According to some embodiments, at least 65% of the activated EVs have a size of above 150 nm. According to some embodiments, at least 70% of the activated EVs have a size of above 150 nm. According to some embodiments, from 25 to 70% of the activated EVs have a size of above 150 nm. According to other embodiments, from 25 to 35% of the activated EVs have a size of above 150 nm. According to certain embodiments, from 25 to 45% of the activated EVs have a size of above 150. According to some embodiments, from 30 to 70% of the activated EVs have a size of above 150 nm. According to one embodiment, from 35 to 65% of the activated EVs have a particle diameter size of above 150 nm. According to another embodiment, from 35 to 45% of the activated EVs have a size of above 150 nm. According to yet another embodiment, from 32 to 65% of the activated EVs have a size of above 150 nm.

The term “X nm or more” encompasses also the term “more than X nm” and may be replaced by it in any one of the embodiments of the present invention.

According to some embodiments, at least 0.2%, or at least 0.5% or at least 0.8% of the isolated activated EV have the size above 300 nm, e.g. between 300 to 600 nm. According to some embodiments, from about 0.3 to about 3% of the isolated activated EV have the size between 300 to 500 nm.

According to some embodiments, the mean size of the activated EVs is 130 nm or more, as measured by nanoparticle-tracking analysis (NTA). According to other embodiments, the mean size of the activated EVs is 132 nm or more. According to other embodiments, the mean size of the activated EVs is 135 nm or more. According to other embodiments, the mean size of the activated EVs is 137 nm or more. According to other embodiments, the mean size of the activated EVs is 140 nm or more. According to other embodiments, the mean size of the activated EVs above 140 nm. According to other embodiments, the mean size of the activated EVs is 142 nm or more. According to other embodiments, the mean size of the activated EVs is 145 nm or more. According to other embodiments, the mean size of the activated EVs is 147 nm or more. According to other embodiments, the mean size of the activated EVs is 150 nm or more. According to other embodiments, the mean size of the activated EVs is 152 nm or more. According to other embodiments, the mean size of the activated EVs is 155 nm or more. According to other embodiments, the mean size of the activated EVs is 160 nm or more. According to other embodiments, the mean size of the activated EVs above 160 nm. According to other embodiments, the mean size of the activated EVs is 162 nm or more. According to other embodiments, the mean size of the activated EVs is 165 nm or more. According to other embodiments, the mean size of the activated EVs is above 170 nm.

According to some embodiments, the present invention provides isolated activated extracellular vesicles (EVs) derived from pre-stimulated T-cells expressing a chimeric antigen receptor (CAR T-cells), wherein at least 25% of the EVs have a size of above 150 nm and the EVs have the mean size of 135 or more. According to other embodiments, the present invention provides isolated activated extracellular vesicles (EVs) derived from activated T-cells expressing a chimeric antigen receptor (CAR T-cells), wherein at least 25% of the EVs have a size of above 150 nm, and the EVs have the mean size of 140 or more. According to some embodiments, at least 25% of the EVs have a size of above 150 nm and the EVs have the mean size of 140 nm or more, 145 nm or more, 147 nm or more, 150 nm or more, 155 nm or more, 160 nm or more or 170 nm or more. According to some embodiments, the present invention provides isolated activated extracellular vesicles (EVs) derived from activated T-cells expressing a chimeric antigen receptor (CAR T-cells), wherein at least 25% of the EVs have a size of above 150 nm and the EVs have the mean size of 147 nm or more. According to some embodiments, the present invention provides isolated activated extracellular vesicles (EVs) derived from activated T-cells expressing a chimeric antigen receptor (CAR T-cells), wherein at least 25% of the EVs have a size of above 150 nm and the EVs have the mean size of 155 nm or more. According to some embodiments, at least 25% of the EVs have a size of above 150 nm and the EVs have the mean size of 160 nm or more. According to some embodiments, at least 25% of the EVs have a size of above 150 nm and the EVs have the mean size of 170 nm or more. According to some embodiments, at least 29% of the EVs have a size of above 150 nm and the EVs have the mean size of 140 nm or more, 145 nm or more, 147 nm or more, 150 nm or more, 155 nm or more, 160 nm or more or 170 nm or more. According to some embodiments, at least 29% of the EVs have a size of above 150 nm and the EVs have the mean size of 140 nm or more, 145 nm or more, 147 nm or more, 150 nm or more, 155 nm or more, 160 nm or more, 162 nm or more or 170 nm or more. According to some embodiments, at least 29% of the EVs have a size of above 150 nm and the EVs have the mean size of 147 nm or more. According to some embodiments, at least 29% of the EVs have a size of above 150 nm and the EVs have the mean size of 150 nm or more. According to some embodiments, at least 29% of the EVs have a diameter size of above 150 nm and the EVs have the mean size of 155 nm or more. According to some embodiments, at least 29% of the EVs have a size of above 150 nm and the EVs have the mean size of 160 nm or more. According to some embodiments, at least 29% of the EVs have a size of above 150 nm and the EVs have the mean size of 165 nm or more. According to some embodiments, at least 30% of the EVs have a size of above 150 nm and the EVs have the mean size of 140 nm or more, 145 nm or more, 147 nm or more, 150 nm or more, 155 nm or more, 160 nm or more or 170 nm or more. According to some embodiments, at least 32% of the EVs have a size of above 150 nm and the EVs have the mean size of 140 nm or more, 145 nm or more, 147 nm or more, 150 nm or more, 155 nm or more, 160 nm or more or 170 nm or more. According to some embodiments, at least 35% of the EVs have a size of above 150 nm and the EVs have the mean size of 140 nm or more, 145 nm or more, 147 nm or more, 150 nm or more, 155 nm or more, 160 nm or more or 170 nm or more. According to some embodiments, at least 40% of the EVs have a size of above 150 nm and the EVs have the mean size of 140 nm or more, 145 nm or more, 147 nm or more, 150 nm or more, 155 nm or more, 160 nm or more, 162 nm or more or 170 nm or more. According to some embodiments, at least 40% of the EVs have a size of above 150 nm and the EVs have the mean size of 147 nm or more. According to some embodiments, at least 40% of the EVs have a size of above 150 nm and the EVs have the mean size of 155 nm or more. According to some embodiments, at least 40% of the EVs have a size of above 150 nm and the EVs have the mean size of 160 nm or more. According to some embodiments, at least 40% of the EVs have a size of above 150 nm and the EVs have the mean size of 165 nm or more. According to some embodiments, at least 40% of the EVs have a size of above 150 nm and the EVs have the mean size above 170. According to some embodiments, at least 40% of the EVs have a size of above 150 nm and the EVs have the mean size of 170 nm or more. According to some embodiments, at least 43% of the EVs have a size of above 150 nm and the EVs have the mean size of 140 nm or more, 145 nm or more, 147 nm or more, 150 nm or more, 155 nm or more, 160 nm or more or 170 nm or more. According to some embodiments, at least 46% of the EVs have a size of above 150 nm and the EVs have the mean size of 140 nm or more, 145 nm or more, 147 nm or more, 150 nm or more, 155 nm or more, 160 nm or more or 170 nm or more. According to some embodiments, at least 46% of the EVs have a size of above 150 nm and the EVs have the mean size of 147 nm or more. According to some embodiments, at least 50% of the EVs have a size of above 150 nm and the EVs have the mean size of 147 nm or more or of 150 nm or more or of 155 nm or more. According to some embodiments, at least 60% of the EVs have a size of above 150 nm and the EVs have the mean size of 150 nm or more or of 155 nm or more or of 160 nm or more.

According to some embodiments, at least 0.5%, at least 1%, at least 1.5%, at least 2% or at least 3% have size of above 300 nm.

According to some embodiments, the ratio between EVs having the particle size of above 150 nm and EVs having the particle size of below 150 nm is from 2:8 to 8:8. According to some embodiments, the ratio between EVs having the particle size of above 150 nm and EV having the particle size of below 150 nm is about 2:8, about 3:8, about 4:8, about 5:8, about 6:8, about 7:8 or about 1:1. According to some embodiments, the ratio between EVs having the particle size of above 150 nm and EV having the particle size of below 150 nm is about 1:3, about 2:3, or about 1:1. According to some embodiments, at least 25% of the EVs have a size of above 150 nm, the EVs have the mean size of 132 nm or more, 135 nm or more, 137 nm or more, 140 nm or more, 145 nm or more, 147 nm or more, 150 nm or more, 155 nm or more, 160 nm or more or 170 nm or more and the ratio between EVs having the size of above 150 nm and EV having the size of below 150 nm is from 1:4 to 1:1 or about 1:4, about 1:3, about 2:3, or about 1:1.

According to some embodiments, at least 20%, at least 25%, at least 30%, at least 35%, at least 40%, at least 45%, at least 50%, at least 55%, or at least 60% of the EVs have size above 155 nm, or above 160 nm.

The terms “derived from” and “originated from” are used herein interchangeably and refer to extracellular vesicles that are produced within, by, or from, a specified cell, cell type, or population of cells such as T-cells, and in particular from CART cells.

As used herein, the terms “parent cell”, “producer cell” and “original cell” include any cell from which the extracellular vesicle is derived and isolated. The terms also encompasses a cell that shares a protein, lipid, sugar, or nucleic acid component of the extracellular vesicle. For example, a “parent cell” or “producer cell” include a cell which serves as a source for the extracellular vesicle membrane. The term “original CAR T-cells” subsequently refers to CAR T-cells from which the EVs are derived.

The terms “purify,” “purified,” “purifying”, “isolate”, “isolated,” and “isolating” are used herein interchangeably and refer to the state of a population (e.g., a plurality of known or unknown amount and/or concentration) of extracellular vesicles, that have undergone one or more processes of purification/isolation, e.g., a selection of the desired extracellular vesicles, or alternatively a removal or reduction of residual biological products and/or removal of undesirable extracellular vesicles, e.g. removing EVs having a particular size. According to one embodiment, the ratio of EVs to residual parent cells is at least 2, 3, 4, 5, 6, 8 or 10 times higher, or in certain advantageous embodiments at least 50, 100, 1000, or 2000 times higher than in the initial material. According to some embodiments, the ratio is weight ratio. In some advantageous embodiments, the term “isolated” have the meaning of substantially cell-free or cell-free, and may be substituted by it.

The terms “chimeric antigen receptor” and “CAR” are used herein interchangeably and refer to an engineered receptor composed of heterologous domains, which include at least an extracellular antigen-binding domain, a transmembrane domain, and a cytoplasmic signaling domain capable of activating T cells.

The extracellular portion or domain of a CAR comprises an antigen binding domain and optionally a spacer or hinge region. The antigen binding domain of the CAR targets and specifically binds to an antigen of interest, e.g. a tumor-associated antigen (TAA). The targeting regions may comprise full length heavy chain, Fab fragments, or single chain variable fragment (scFvs). The antigen binding domain can be derived from the same species or from a different species than the one in which the CAR will be used in. In one embodiment, the antigen binding domain is a scFv.

The extracellular spacer or hinge region of a CAR is located between the antigen binding domain and a transmembrane domain. Extracellular spacer domains may include, but are not limited to, Fc fragments of antibodies or fragments or derivatives thereof, hinge regions of antibodies or fragments or derivatives thereof, CH2 regions of antibodies, CH3 regions of antibodies, accessory proteins, artificial spacer sequences or combinations thereof.

The term “transmembrane domain” refers to the region of the CAR, which crosses or bridges the plasma membrane. The transmembrane domain of the CAR of the invention is the transmembrane region of a transmembrane protein, an artificial hydrophobic sequence or a combination thereof.

The term “intracellular domain” refers to the intracellular part of the CAR comprising an activation domain capable of activating T cells and optionally additional co-stimulatory domain(s). The intracellular domain may be an intracellular domain of a T cell receptor (e.g. the zeta chain associated with the T cell receptor complex) and/or may comprise stimulatory domains of other receptor (e.g., TNFR superfamily members) or portion thereof, such as an intracellular activation domain (e.g., an immunoreceptor tyrosine-based activation motif (ITAM)-containing T cell activating motif), an intracellular costimulatory domain, or both. According to some embodiments, the costimulatory domain is selected from a costimulatory domain of CD28, 4-1BB, OX40, iCOS, CD27, CD80 and CD70. According to one embodiment, the costimulatory domain is a costimulatory domain of CD28. According to one another, the costimulatory domain a costimulatory domain of 4-1BB.

The terms “binds specifically” or “specific for” with respect to an antigen-binding domain of a CAR or of an antibody, of a fragment thereof refers to an antigen-binding domain which recognizes and binds to a specific antigen, but does not substantially recognize or bind other molecules in a sample. The term encompasses that the antigen-binding domain binds to the antibody-recognizing portion of its antigen (epitope) with high affinity, and does not bind to other unrelated epitopes with high affinity.

The CAR may be specific in some embodiments to a tumor associated antigen. The terms “tumor associated antigen” and “TAA” are used herein interchangeably and refer to any antigen, which is found in significantly higher concentrations in or on tumor cells than on normal cells. According to any one of the above embodiments, the CAR of the CAR T-cells specifically binds to a tumor associated antigen (TAA). Any CAR that binds to a TAA may be used according to the teaching of the present invention. According to one embodiment, the TAA is HER2. According to another embodiment, the CAR of the CAR T-cell specifically binds to a tumor associated antigen selected from CD19, CD38 and CD24. Other non-limiting examples of CARs that may be used are CAR against an antigen selected from MUC1, Mesothelin, PSCA, EGFR, EPCAM, CEA, PSMA, GPC3, LMP1, CD133, cMET, GD2, HER2, ROR1, CD70, CD38, CD138, CD24, and CD19.

The term “T cell” as used herein refers a lymphocyte that expresses T-cell receptors and participates in a variety of cell-mediated immune reactions, as well known in art. T cells may include CD4⁺ T-cells, CD8⁺ T-cells and natural killer T-cells. The term encompasses genetically modified T-cells, e.g. transduced with a nucleic acid such as DNA or RNA, optionally using a vector. The term “CAR T cell” refers to a T-cell expressing a CAR. In certain embodiments, the invention relates to CAR T cells comprising a population of CD8⁺ T-cells.

As described above, the activated EVs are considered to comprise at least a part of the molecular contents of the parental cells. According to some embodiments, the activated EVs of the present invention comprise the chimeric antigen receptor (CAR) of the original CAR T-cell (in at least a subset of the EVs population as disclosed herein). According to one embodiment, at least 1%, at least 2%, at least 3%, at least 4%, at least 5%, at least 7%, at least 10%, at least 15%, at least 20% or at least 25% of the activated EVs of the present invention comprise the chimeric antigen receptor (CAR) of the original CAR T-cell. According to one embodiment, the CAR is presented on the outer membrane of the EVs. According to some embodiments, at least 10% of the isolated activated EVs of the present invention comprise the chimeric antigen receptor. According to some embodiments, at least 15% of the isolated activated EVs of the present invention comprise the chimeric antigen receptor. According to some embodiments, at least 18% of the isolated activated EVs of the present invention comprise the chimeric antigen receptor. According to some embodiments, at least 20% of the isolated activated EVs of the present invention comprise the chimeric antigen receptor. According to some embodiments, at least 3%, at least 5%, at least 10% of the isolated activated EVs of the present invention present the chimeric antigen receptor. According to some embodiments, at least 15% of the isolated activated EVs of the present invention present the chimeric antigen receptor. According to some embodiments, at least 25% or at least 30% of the isolated activated EVs of the present invention comprise the chimeric antigen receptor. According to some embodiments, from 10 to 90% of EVs present the CAR. According to some embodiments, from 15 to 85%, from 20 to 80, from 25% to 75%, from 30 to 60%, from 20 to 60%, from 20 to 50%, from 15 to 45%, from 15 to 40% of the EVs present the CAR. According to some embodiments, the CAR is anti-HER2 CAR. According to one embodiment, the CAR is N29 CAR.

The terms “HER2” and “human HER2” are used herein interchangeably and refer to the protein known as human epidermal growth factor receptor 2, receptor tyrosine-protein kinase erbB-2, also known as CD340 (cluster of differentiation 340), proto-oncogene Neu, Erbb2 (rodent), or ERBB2 and has an extension number EC 2.7.10.1. The terms “anti Her2” or “αHer2” refers to an antigen binding domain of a CAR or of an antibody that binds specifically to human Her2.

According to some embodiments, the EVs of the present invention are originated from CAR T cell, wherein the CAR binds specifically to HER2 (anti-HER2 CAR). According to one embodiment, the anti-HER2 CAR comprises 3 complementarity determining regions (CDRs) of a light variable chain having amino acid sequence SEQ ID NO: 1 and 3 CDRs of a heavy variable chain having amino acid sequence SEQ ID NO: 2. According to one embodiment, the CAR that binds specifically to HER2 is N29 CAR, as known in the art, e.g. as described in Globerson-Levin A, et al., Molecular therapy, 2014; 22(5); 1029-38. In general, N29 is a monoclonal antibody binding specifically human HER2 receptor, and N29 CAR comprises a scFv of said N29 antibody as an antigen binding domain. According some embodiments, the CAR T-cells express N29 CAR (N29 CAR T-cells). According to one embodiment, the activated EVs are derived from activated N29 CAR T-cells. According to some embodiments, the N29 CAR has amino acid sequence SEQ ID NO: 3. According to other embodiments, the N29 CAR is encoded by DNA sequence SEQ ID NO: 4.

According to any one of the above embodiments, the CAR T-cells are activated CAR T-cells. The terms “pre-stimulated”, “pre-activated”, “stimulated” and “activated” with respect to CAR T-cells are used herein interchangeably and refer to CAR T-cells that have been incubated and therefore stimulated with a tumor associated antigen to which the CAR binds specifically. Furthermore the term refers to a state of the T-cells provided with a CAR-mediated stimulation prior to EVs isolation. Such activated CAR T cells may also be denoted as “specifically activated CAR T cell” and the EVs obtained from the activated CAR T cells are denoted as activated EVs, although the term activated with respect to EVs may be omitted in some of the embodiments. The activation is effected (performed) by incubation of CAR T cells with a specific tumor associated antigen for a period of time sufficient to activate the T-cells, as known in the art.

According to some embodiments, the incubation is performed for from 3 to 96, from 6 to 72, or from 12 to 48 hours, e.g. for 24 hours. Activation can for example be associated with induced cytokine production, elevation levels of IL-2, IL-5, IL-8, IL-12, IL-17, IL-21, MCP-1 (CCL2), MIP—1α (CCL3), MIP-1β (CCL4), RANTES (CCL5), MIG (CXCL9), IP10 (CXCL10), fractalkine (CX3CL1), G-CSF, GM-CSF, Flt-3L, IL-1Rα, and/or TNFα, elevates expression of receptors such as CD25 (the IL-2 receptor) and CD71 (the transferrin receptor), elevates expression of co-stimulatory molecules such as CD26, CD27, CD28, CD30, CD154 or CD40L, and CD134, and detectable effector functions. With respect to T-cells, “activation” may have also the meaning of the state of a T cell that has been sufficiently stimulated to induce detectable cellular proliferation.

The terms “stimulated” and “activated” with respect to EVs means that the EVs have been obtained from activated CAR T-cells as described above. Activated EVs as described herein typically manifest improved properties (e.g. anti-cancer properties characteristic of their parent CAR T-cells) compared to native, non-activated EVs (EVs obtained from non-activated CAR T-cells). As discussed above, the activated EVs may include the content or the partial content of their parent CAR T-cells. Thus, in some embodiments the EVs comprise or express elevated levels of cytokines and/or receptors as in their parent activated CAR T-cells. Without wishing to be bound by a specific theory or mechanism of action, activated EVs may be distinguished from non-activated EVs by the presence of surface markers and/or intracellular markers. For example, without limitation, activated EVs may contain or express increased levels of T cell activation markers, e.g. CD25 and/or CD137 (41-BB), CD3, CD38 and HLARD.

According to some embodiments, at least 15% of the EVs of the present invention express CD3 antigen on their outer membrane. According to some embodiments at least 20%, at least 22%, at least 24%, at least 25%, or at least 28% of the EVs of the present invention express CD3 antigen on their outer membrane. According to some embodiments, at least 25% of the EVs of the present invention express CD3 antigen on their outer membrane.

According to some embodiments, at least 20% of the EVs of the present invention express HLARD antigen on their outer membrane. According to some embodiments, at least 22%, at least 24%, at least 25%, or at least 28% of the EVs of the present invention express HLARD antigen on their outer membrane. According to some embodiments, at least 25% of the EVs of the present invention express HLARD antigen on their outer membrane.

According to some embodiments, at least 10% of the EVs of the present invention express CD38 antigen on their outer membrane. According to some embodiments at least 8%, at least 12%, at least 15%, at least 18%, or at least 20% of the EVs of the present invention express CD38 antigen on their outer membrane. According to some embodiments, at least 25% of the EVs of the present invention express CD38 antigen on their outer membrane.

According to some embodiments, at least 15% of the EVs of the present invention express CD3 antigen on their outer membrane, at least 20% of the EVs of the present invention express HLARD antigen on their outer membrane and at least 8% of the EVs of the present invention express CD38 antigen on their outer membrane.

According to the teaching of the present invention, activated EVs are those obtained from CAR T-cells activated by incubation with their corresponding TAA, i.e. a TAA to which the CAR binds specifically and consequently activates the T-cells. According to one embodiment, the CAR-T cells were incubated with TAA from 6 to 48 or from 12 to 36 hours. In other embodiments, the T cells were provided with a CAR-mediated stimulation no more than 24 hours prior to EV collection, e.g. up to 18, 12 or 6 hours prior to EVs isolation. According to certain exemplary embodiments, the T cells have been activated by cells (e.g. tumor cells or antigen-presenting cells) presenting the TAA. According to certain exemplary embodiments, the T cells are activated by TAA expressed or presented by an entity such as liposomes or TAA attached to a surface of an entity such as a plate. According to certain exemplary embodiments, the T cells are activated by a surface presenting the TAA to which the CAR binds specifically. According to one embodiment, the TAA is HER2 and the CAR is N29 CAR. According to another embodiment, the CAR is N29 CAR and the CAR T-cells were incubated with ovarian cancer cells expressing HER2. According to one embodiment, the ovarian cancer cells are SKOV cells. According to another embodiment, the CAR is N29 CAR and the CAR T-cells are incubated with HER2 positive breast cancer cells. According to another embodiment, the CAR is N29 CAR and the CAR T-cells are incubated with HER2 antigen. According to some embodiments, the EVs are derived from activated N29 CAR T-cells. According to one embodiment, the EVs are derived from N29 CAR T-cells activated with HER2 positive ovarian cancer cells. According to one embodiment, the EVs are derived from N29 CAR T-cells activated with SKOV cells. According to one embodiment, the EVs are derived from N29 CAR T-cells activated with HER2 breast cancer cells. According to one embodiment, the EVs are derived from N29 CAR T-cells activated with primary HER2 positive cancer cells. According to one embodiment, the primary HER2 positive cancer cells are cells obtained from a subject suffering from said cancer. According to some embodiments, the T-cells are CD8⁺ T-cells. According to other embodiments, the T-cells are CD4⁺ T-cells. According to yet another embodiment, the CAR T-cells are a combination of CD4⁺ and CD8⁺ CAR T-cell.

According to some embodiments, the present invention provides extracellular vesicles (EVs), wherein the EVs are derived from activated N29 CAR T-cells, wherein at least 20% of the EVs have size of above 150 nm. According to some embodiments, the present invention provides extracellular vesicles (EVs), wherein the EVs are derived from activated N29 CAR T-cells, wherein at least 22% of the EVs have size of above 150 nm. According to some embodiments, the present invention provides extracellular vesicles (EVs), wherein the EVs are derived from activated N29 CAR T-cells, wherein at least 25% of the EVs have size of above 150 nm. According to some embodiments, at least 27% of the activated EVs have a size of 150 nm or more. According to one embodiment, at least 28% of the activated EVs have a size of 150 nm or more. According to one embodiment, at least 29% of the activated EVs have a size of 150 nm or more. According to one embodiment, at least 30% of the activated EVs have a size of 150 nm or more. According to one embodiment, at least 32% of the activated EVs have a size of 150 nm or more. According to one embodiment, at least 35% of the activated EVs have a size of 150 nm or more. According to another embodiment, at least 40% of the activated EVs have a size of above 150 nm. According to another embodiment, at least 42% of the activated EVs have a size of above 150 nm. According to yet another embodiment, at least 45% of the activated EVs have a size of above 150 nm. According to another embodiment, at least 50% of the activated EVs have a size of above 150 nm. According to another embodiment, at least 55% of the activated EVs have a size of above 150 nm. According to yet another embodiment, at least 60% of the activated EVs have a size of above 150 nm. According to yet another embodiment, at least 65 or at least 65% of the activated EVs have a size of above 150 nm. According to some embodiments, from 22 to 70% or from 25 to 70% of the activated EVs have a size of above 150 nm. According to other embodiments, from 25 to 35% of the activated EVs have a size of above 150 nm. According to certain embodiments, from 25 to 45% of the activated EVs have a size of above 150. According to some embodiments, from 30 to 70% of the activated EVs have a size of above 150 nm. According to one embodiment, from 35 to 65% of the activated EVs have a size of above 150 nm. According to other embodiments, the mean size of the activated EVs is 135 nm or more. According to other embodiments, the mean size of the activated EVs is 140 nm or more. According to some embodiments, the mean size of the activated EVs is 145 nm or more. According to certain embodiments, the mean size of the activated EVs is 150 nm or more. According to one embodiment, the mean size of the activated EVs is 155 nm or more, 160 nm or more, or 165 nm or more. According to one embodiment, the mean size of the activated EVs is above 160 nm or above 170 nm. According to some embodiments, the present invention provides isolated activated EVs derived from activated N29 CAR T-cells, wherein at least 22%, at least 25%, at least 29%, at least 30%, at least 32%, at least 35%, at least 37%, at least 40%, at least 43%, at least 45% or at least 46%, at least 50%, at least 55%, at least 60% or at least 65% of the EVs have a particle diameter size of above 150 nm and the EVs have the mean size selected from 132 or more, 135 nm or more, 137 nm or more, 140 nm or more, 142 nm or more, 145 nm or more, 147 nm or more, 150 nm or more, 152 nm or more, 155 nm or more, 160 nm or more, 162 nm or more, 165 nm or more, 167 nm or more and 170 nm or more. According to some embodiments, the present invention provides isolated EVs derived from activated N29 CAR T-cells, wherein at least 25% of the EVs have a size of above 150 nm and the EVs have the mean size selected from 132 or more, 135 nm or more, 137 nm or more, 140 nm or more, 142 nm or more, 145 nm or more, 147 nm or more, 150 nm or more, 152 nm or more, 155 nm or more, 160 nm or more, 162 nm or more, 165 nm or more and above 170 nm. According to some embodiments, the present invention provides isolated activated EVs derived from activated N29 CAR T-cells, wherein at least 29% or at least 30% of the EVs have a size of above 150 nm and the EVs have the mean size selected from 132 or more, 135 nm or more, 137 nm or more, 140 nm or more, 142 nm or more, 145 nm or more, 147 nm or more, 150 nm or more, 152 nm or more, 155 nm or more, 160 nm or more, 162 nm or more, 165 nm or more and above 170 nm. According to some embodiments, the present invention provides isolated activated EVs derived from activated N29 CAR T-cells, wherein at least 35% of the EVs have a size of above 150 nm and the EVs have the mean size selected from 132 or more, 135 nm or more, 137 nm or more, 140 nm or more, 142 nm or more, 145 nm or more, 147 nm or more, 150 nm or more, 152 nm or more, 155 nm or more, 160 nm or more, 162 nm or more, 165 nm or more and above 170 nm. According to some embodiments, the present invention provides isolated activated EVs derived from activated N29 CAR T-cells, wherein at least 22% of the EVs have a size of above 150 nm and the EVs have the mean size of 135 or more. According to some embodiments, the present invention provides isolated activated EVs derived from activated N29 CAR T-cells, wherein at least 25% of the EVs have a size of above 150 nm and the EVs have the mean size of 135 or more. According to other embodiments, the present invention provides isolated activated EVs derived from activated N29 CAR T-cells, wherein at least 25% of the EVs have a size of above 150 nm, and the EVs have the mean size of 140 or more. According to some embodiments, at least 29% of the EVs have a size of above 150 nm and the EVs have the mean size of 147 nm or more. According to some embodiments, at least 29% of the EVs have a size of above 150 nm and the EVs have the mean size of 150 nm or more. According to some embodiments, at least 29% of the EVs have a size of above 150 nm and the EVs have the mean size of 155 nm or more. According to some embodiments, at least 29% of the EVs have a size of above 150 nm and the EVs have the mean size of 160 nm or more. According to some embodiments, at least 29% of the EVs have a size of above 150 nm and the EVs have the mean size of 165 nm or more. According to some embodiments, the present invention provides isolated activated EVs derived from activated N29 CAR T-cells, wherein at least 25% of the EVs have a size of above 150 nm and the EVs have the mean size of 147 nm or more. According to some embodiments, at least 35% of the EVs derived from activated N29 CAR T-cells have a particle size of above 150 nm and the EVs have the mean size of more than 150 nm or 155 nm or more or 160 nm or more. According to some embodiments, at least 40% of the EVs derived from activated N29 CAR T-cells have a particle size of above 150 nm and the EVs have the mean size of 155 nm or more or 160 nm or more or 165 nm or more. According to some embodiments, at least 45% or at least 60% of the EVs derived from activated N29 CAR T-cells have a particle size of above 150 nm and the EVs have the mean size of 155 nm or more or 160 nm or more or 165 nm or more. According to some embodiments, at least 55% of the EVs derived from activated N29 CAR T-cells have a particle size of above 150 nm and the EVs have the mean size of 155 nm or more or 160 nm or more or 165 nm or more. According to some embodiments, at least 60% of the EVs derived from activated N29 CAR T-cells have a size of above 150 nm and the EVs have the mean size of 155 nm or more or 160 nm or more or 165 nm or more. According to some embodiments, at least 25% of the EVs have a size of above 150 nm, the EVs have the mean size of 132 nm or more, 135 nm or more, 137 nm or more, 140 nm or more, 145 nm or more, 147 nm or more, 150 nm or more, 155 nm or more, 160 nm or more or 170 nm or more and the ratio between EVs having the size of above 150 nm and EV having the size of below 150 nm is from 1:4 to 1:1 or about 1:4, about 1:3, about 2:3, or about 1:1. According to some embodiments, at least 25% or at least 30% of the activated EVs of the present invention comprise the N29 CAR. According to some embodiments, at least 15% of the EVs of the EVs derived from activated N29 CAR T-cells express CD3 antigen on their outer membrane. According to some embodiments, at least 20% of the EVs of the EVs derived from activated N29 CAR T-cells express HLARD antigen on their outer membrane. According to some embodiments, at least 8% of the EVs of the EVs derived from activated N29 CAR T-cells express CD38 antigen on their outer membrane. According to some embodiments, at least 20% of the EVs of the present invention express CD3 antigen on their outer membrane, at least 20% of the EVs of the present invention express HLARD antigen on their outer membrane and at least 10% of the EVs of the present invention express CD38 antigen on their outer membrane. According to some embodiments, the N29 CAR T are activated by incubation with HER2 positive cancer cells. According to some embodiments, the HER2 positive cancer cells are selected from ovarian cancer cells expressing HER2, breast cancer cells expressing HER2 and primary cancer cells expressing HER2. According to some exemplary embodiments, the present invention provides isolated activated extracellular vesicles derived from N29 CAR-T cells incubated from 12 to 36 hours with ovarian cancer cells expressing HER2, wherein the EVs are isolated within 24 hours post incubation. According to another embodiment, the N29 CAR-T cells were incubated from 12 to 36 hours with breast cancer cells expressing HER2, wherein the EVs are isolated within 24 hours post incubation. According to some embodiments, the EVs were isolated within 30, 36, 42, 48, 60, 72, 84, 96 hours after the incubation. According to other embodiments, the EVs are isolated within 1, 2, 3, 4, 5, 6 or 7 days after the incubation. For example, T cells may be incubated at a ratio of T cells to target cells of 1.5:1 to 3:1, e.g. 2:1. According to one embodiment, the T cells are incubated with target cells at a ratio of T cells to target cells of from 15:1 to 1:5, from 10:1 to 1:4 from 8:1 to 1:3 from 5:1 to 1:2 or from 3:1 to 1:1.

According to any one of the above embodiments, the activated EVs are cytotoxic EVs, i.e. exhibiting target-specific (e.g. tumor-directed) cytotoxicity. According to one embodiment, the activated EVs exhibit cytotoxic activity toward cancer cells. According to a further embodiment, the activated EVs of the present invention exhibit cytotoxic activity specifically toward cancer cells (e.g. toward those exhibiting or expressing the TAA to which the CAR of the parent cells is directed). According to some embodiments, the cytotoxic activity is apoptosis.

The isolated activated EVs of the invention have been unexpectedly found to exert outstanding anti-tumor effects even in the absence of an exogenously added anti-cancer agent or payload. Thus, in other embodiments, the invention relates to isolated activated EVs of the invention devoid of any exogenous anti-cancer agent.

According to another embodiment, the activated EVs further comprise an anticancer agent. The terms “anti-cancer”, “anti-neoplastic” and “anti-tumor” when referred to a compound, an agent, moiety or a composition are used herein interchangeably and refer to a compound, drug, antagonist, inhibitor, or modulator having anticancer properties or the ability to inhibit or prevent the growth, function or proliferation of and/or causing destruction of cells, and in particular tumor cells. According to some embodiments, the anti-cancer agent is selected from chemotherapeutic agents, radioactive isotopes, toxins, cytokines such as interferons, and antagonistic agents targeting cytokines, cytokine receptors or antigens associated with tumor cells. In some embodiments, an anti-cancer agent is a chemotherapeutic. The term “exogenous anti-cancer agent” as used herein refers to anti-cancer agent that was loaded into the EVs after their isolation from the T-cells.

The present invention provides a formulation, a preparation or a composition comprising a plurality of the isolated activated EVs according the present invention. According to one embodiment, the composition is a pharmaceutical composition. Thus, according to another aspect, the present invention provides a pharmaceutical composition comprising the isolated EVs derived from activated T-cells expressing a chimeric antigen receptor (CAR T-cells) wherein at least 20% of the EVs have a particle size of above 150 nm, and a pharmaceutically acceptable carrier. According to some embodiments, the present invention provides a pharmaceutical composition comprising the isolated activated EVs derived from activated T-cells expressing a chimeric antigen receptor (CAR T-cells) wherein at least 22% of the EVs have size of above 150 nm, and a pharmaceutically acceptable carrier. According to some embodiments, the present invention provides a pharmaceutical composition comprising the isolated activated EVs derived from activated T-cells expressing a chimeric antigen receptor (CAR T-cells) wherein at least 25% of the EVs have size of above 150 nm, and a pharmaceutically acceptable carrier.

Each and every embodiment related to isolated activated EVs as described in any one of the above aspects applies herein as well.

According to any aspect or embodiment of the present invention the term “at least X % of EVs having size above 150 nm” may be replaced by the term “on average X % or more of EVs have size above 150 nm”. Thus, according to some embodiments, the present invention provides isolated activated extracellular vesicles (EVs) derived from activated T-cells expressing a chimeric antigen receptor (CAR T-cells), wherein on average 22% or more of the EVs have a particle size diameter of above 150 nm. According to some embodiments, on average 25% or more, 30% or more, 35% or more, or 40% or more of the EVs have a particle size diameter of above 150 nm.

The term “pharmaceutical composition” as used herein refers to a composition comprising a therapeutic agent (such as activated and isolated EVs of the present invention) formulated together with one or more pharmaceutically acceptable carriers.

The term “therapeutically effective amount” of EVs is an amount of EVs that, when administered to a subject will have the intended therapeutic effect. The therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, the nature and extent of the cognitive impairment, and the therapeutics or combination of therapeutics selected for administration, and the mode of administration. The skilled worker can readily determine the effective amount for a given situation by routine experimentation.

Formulation of pharmaceutical compositions may be adjusted according to applications. In particular, the pharmaceutical composition may be formulated using a method known in the art so as to provide rapid, continuous or delayed release of the active ingredient after administration to mammals. For example, the formulation may be any one selected from among plasters, granules, lotions, liniments, lemonades, aromatic waters, powders, syrups, ophthalmic ointments, liquids and solutions, aerosols, extracts, elixirs, ointments, fluidextracts, emulsions, suspensions, decoctions, infusions, ophthalmic solutions, tablets, suppositories, injections, spirits, capsules, creams, troches, tinctures, pastes, pills, and soft or hard gelatin capsules. According to one embodiment, the pharmaceutical composition is a liquid composition. According to another embodiment, the composition is an injectable composition.

The term “pharmaceutically acceptable carrier” or “pharmaceutically acceptable excipient” as used herein refers to any and all solvents, dispersion media, preservatives, antioxidants, coatings, isotonic and absorption delaying agents, surfactants, fillers, disintegrants, binders, diluents, lubricants, glidants, pH adjusting agents, buffering agents, enhancers, wetting agents, solubilizing agents, surfactants, antioxidants the like, that are compatible with pharmaceutical administration. The use of such media and agents for pharmaceutically active substances is well known in the art. The compositions may contain other active compounds providing supplemental, additional, or enhanced therapeutic functions. Solid carriers or excipients such as, for example, lactose, starch or talcum or liquid carriers such as, for example, water, fatty oils or liquid paraffins.

Other carriers or excipients which may be used include, but are not limited to, materials derived from animal or vegetable proteins, such as the gelatins, dextrins and soy, wheat and psyllium seed proteins; gums such as acacia, guar, agar, and xanthan; polysaccharides; alginates; carboxymethylcelluloses; carrageenans; dextrans; pectins; synthetic polymers such as polyvinylpyrrolidone; polypeptide/protein or polysaccharide complexes such as gelatin-acacia complexes; sugars such as mannitol, dextrose, galactose and trehalose; cyclic sugars such as cyclodextrin; inorganic salts such as sodium phosphate, sodium chloride and aluminium silicates; and amino acids having from 2 to 12 carbon atoms and derivatives thereof such as, but not limited to, glycine, L-alanine, L-aspartic acid, L-glutamic acid, L-hydroxyproline, L-isoleucine, L-leucine and L-phenylalanine. Each possibility represents a separate embodiment of the present invention.

Solutions or suspensions used for parenteral, intradermal, or subcutaneous application typically include the following components: a sterile diluent such as water for injection, saline solution, fixed oils, polyethylene glycols, glycerine, propylene glycol (or other synthetic solvents), antibacterial agents (e.g., benzyl alcohol, methyl parabens), antioxidants (e.g., ascorbic acid, sodium bisulfate), chelating agents (e.g., ethylenediaminetetraacetic acid), buffers (e.g., acetates, citrates, phosphates), and agents that adjust tonicity (e.g., sodium chloride, dextrose). The pH can be adjusted with acids or bases, such as hydrochloric acid or sodium hydroxide, for example. The parenteral preparation can be enclosed in ampules, disposable syringes or multiple dose glass or plastic vials.

Pharmaceutical compositions adapted for parenteral administration include, but are not limited to, aqueous and non-aqueous sterile injectable solutions or suspensions, which can contain antioxidants, buffers, bacteriostats and solutes that render the compositions substantially isotonic with the blood of an intended recipient. Such compositions can also comprise water, alcohols, polyols, glycerine and vegetable oils, for example. Extemporaneous injection solutions and suspensions can be prepared from sterile powders, granules and tablets. Such compositions preferably comprise a therapeutically effective amount of a compound of the invention and/or other therapeutic agent(s), together with a suitable amount of carrier so as to provide the form for proper administration to the subject.

The terms “pharmaceutically acceptable” and “pharmacologically acceptable” include molecular entities and compositions that do not produce an adverse, allergic, or other untoward reactions when administered to an animal, or human, as appropriate. For human administration, preparations should meet sterility, pyrogenicity, general safety, and purity standards as required by a government drug regulatory agency, e.g., the United States Food and Drug Administration (FDA) Office of Biologics standards.

The terms “enrich”, “enriched” or “enriching” are used interchangeably and refer to a composition comprising higher content and/or concentration of extracellular vesicles than the initial composition. In some embodiments, the composition or the pharmaceutical composition of the present invention are enriched compositions, i.e. has the amount and/or concentration of extracellular vesicles higher that the initial amount and/or concentration obtained upon purification of the EVs. In some embodiments, the concentration of activated EVs is 1.1, 1.5, 2, 3, 5, 10, 50, 100, 500 or 1000 times higher compared to the starting material.

According to one embodiment, the pharmaceutical composition comprises activated and isolated EVs derived from activated CAR T-cells. According to one embodiment, the CAR T-cells were activated by incubation with the TAA to which the CAR binds specifically. According to one embodiment, the CAR T-cells were incubated with the TAA from 1 to 96 hour. According to some embodiments, the CAR T-cells were incubated with TAA from 8 to 48 or from 12 to 36 hour. According to one embodiment, the TAA is HER2 and the CAR is N29 CAR. According to another embodiment, the CAR is N29 CAR and the CAR T-cells were incubated with ovarian cancer cells presenting HER2. According to a further embodiment, the CAR is N29 CAR and the CAR T-cells were incubated with breast cancer cells presenting HER2. According to one embodiment, the ovarian cancer cells are SKOV cells. According to a further embodiment, the CAR is N29 CAR and the CAR T-cells are N29 CAR T-cells incubated with primary HER2 positive cells.

According to some embodiments, the present invention provides a pharmaceutical composition comprising the isolated activated EVs derived from activated T-cells expressing an anti-HER2 CAR, and a pharmaceutically acceptable carrier, wherein at least 22 or at least 25% of the EVs have size of above 150 nm. According to some embodiments, the present invention provides a pharmaceutical composition comprising the isolated activated EVs derived from activated N29 CAR T-cells, and a pharmaceutically acceptable carrier, wherein at least 25% of the EVs have size of above 150 nm.

According to some embodiments, the T-cells are selected from CD4+ T-cells, CD8+ T-cells and a combination thereof. According to one embodiment, the CAR T-cells are activated with HER2, e.g. with the ovarian or breast cancer cells presenting HER2.

According to some embodiments, the pharmaceutical composition is devoid of any additional anti-tumor agents. In other embodiments the invention relates to pharmaceutical compositions comprising the isolated activated EVs of the invention as a sole anti-cancer agent.

The terms “substantially devoid”, “essentially devoid”, “devoid”, “does not include” and “does not comprise” may be used interchangeably and refer to composition that does not include, contain or comprise a particular component, e.g. said composition comprises less than 0.1 wt %, less than 0.01 wt %, or less than 0.001 wt % of the component. In some embodiments, the term devoid contemplates composition comprising traces of the devoid component such as traces of a component used in purification process.

According to other embodiments, the composition further comprise an additional anti-cancer agent. According to some embodiments, the anti-cancer agent is selected from chemotherapeutic agents, radioactive isotopes, toxins, cytokines such as interferons, and antagonistic agents targeting cytokines, cytokine receptors or antigens associated with tumor cells. In some embodiments, an anti-cancer agent is a chemotherapeutic agent. In other embodiments, the additional anti-cancer agent is CAR T-cells, wherein the CAR of said T-cells differs from the CAR of the CAR T-cells from which the EVs are originated.

According to some embodiments, the pharmaceutical composition is a cell-free composition.

According to any one of the above embodiment, the pharmaceutical composition of the present invention is for use in treating cancer.

The term “treating cancer” as used herein should be understood to e.g. encompass treatment resulting in a decrease in tumor size; a decrease in rate of tumor growth; stasis of tumor size; a decrease in the number of metastasis; a decrease in the number of additional metastasis; a decrease in invasiveness of the cancer; a decrease in the rate of progression of the tumor from one stage to the next; inhibition of tumor growth in a tissue of a mammal having a malignant cancer; control of establishment of metastases; inhibition of tumor metastases formation; regression of established tumors as well as decrease in the angiogenesis induced by the cancer, inhibition of growth and proliferation of cancer cells and so forth. The term “treating cancer” as used herein should also be understood to encompass prophylaxis such as prevention as cancer reoccurs after previous treatment (including surgical removal) and prevention of cancer in an individual prone (genetically, due to life style, chronic inflammation and so forth) to develop cancer. As used herein, “prevention of cancer” is thus to be understood to include prevention of metastases, for example after surgical procedures or after chemotherapy.

As used herein, the term “cancer” refers to all types of cancer, neoplasm or malignant tumors found in mammals, including leukemias, lymphomas, melanomas, neuroendocrine tumors, carcinomas and sarcomas. According to one embodiment, cancer is a solid tumor. According to one embodiment, cancer is selected from breast cancer, ovarian cancer, lung adenocarcinoma, stomach, mammary carcinomas, melanoma, skin neoplasms, lymphoma, leukemia, gastrointestinal tumors, including colon carcinomas, stomach carcinomas, pancreas carcinomas, colon cancer, small intestine cancer, ovarian carcinomas, cervical carcinomas, lung cancer, prostate cancer, kidney cell carcinomas and/or liver metastases.

According to some embodiments, the cancer is cancer which cells present the antigen to which the CAR binds specifically. According to some embodiments, the cancer present HER2 antigen. According to other embodiments, the cancer present CD19. According to yet another embodiment, the cancer present CD38 antigen.

According to some embodiments, the cancer is selected from breast cancer, ovarian cancer, lung adenocarcinoma, stomach, liver, pancreatic and brain cancers and hematology malignancies.

In one embodiment, the pharmaceutical composition comprising isolated activated EVs derived from activated anti-HER2 CAR T-cells is for use in treating HER2 positive cancer.

According to some embodiments, the HER2 positive cancer is selected from ovarian cancer, breast cancer, stomach cancer, lung adenocarcinoma, uterine cancer, uterine endometrial carcinoma and HER2+ salivary duct carcinoma.

The terms “HER2 positive” and “HER2+” are used herein interchangeably and refer to cells overexpressing HER2 antigen.

According to other embodiments, the pharmaceutical composition comprising isolated activated EVs of the present invention derived from anti-HER2 CAR T-cells activated with HER2 specific activation, is for use in treating HER2 positive ovarian cancer. According to some embodiments, the pharmaceutical composition comprising isolated activated EVs of the present invention derived from activated anti-HER2 CAR T-cells is for use in treating HER2 positive breast cancer. According to alternative embodiments, the pharmaceutical composition comprising isolated activated EVs of the present invention derived from activated anti-HER2 CAR T-cells is for use in treating HER2 positive ovarian cancer. According to some embodiments, anti-HER2 CAR is N29 CAR. According to some embodiments, the pharmaceutical composition comprising activated and isolated EVs of the present invention derived from activated N29 CAR T-cells is for use in treating breast cancer. According to a further embodiment, the pharmaceutical composition comprising isolated activated EVs of the present invention derived from activated N29 CAR T-cells is for use in treating ovarian cancer. According to yet another embodiment, the pharmaceutical composition comprising isolated activated EVs of the present invention derived from activated N29 CAR T-cells is for use in treating lung adenocarcinoma or stomach cancer. According to some embodiments, the pharmaceutical composition comprises isolated activated EVs derived from activated N29 CAR T-cells, wherein at least 22%, at least 25%, at least 29%, at least 30%, at least 32%, at least 35%, at least 37%, at least 40%, at least 43%, at least 45% or at least 46%, at least 50% or at least 55% of the EVs have a particle diameter size of above 150 nm and/or the EVs have the mean size selected from 132 or more, 135 nm or more, 137 nm or more, 140 nm or more, 142 nm or more, 145 nm or more, 147 nm or more, 150 nm or more, 152 nm or more, 155 nm or more, 160 nm or more, 162 nm or more, 165 nm or more and 170 nm or more. According to other embodiments, the present invention provides isolated activated EVs derived from activated N29 CAR T-cells, wherein at least 25% of the EVs have a size of above 150 nm, and the EVs have the mean size of 140 or more. According to some embodiments, at least 29% of the EVs have a size of above 150 nm and the EVs have the mean size of 150 nm or more, of 155 nm or more, of 160 nm or more, or of 165 nm or more. According to some embodiments, the ratio between EVs having the particle size of above 150 nm and EV having the particle size of below 150 nm is from 1:4 to 1:1 or about 1:4, about 1:3, about 2:3, or about 1:1. According to some embodiments, at least 20% of the EVs of the present invention express CD3 antigen on their outer membrane and/or at least 20% of the EVs of the present invention express HLARD antigen on their outer membrane and/or at least 10% of the EVs of the present invention express CD38 antigen on their outer membrane. According to some embodiments, at least 10% or at least 15% or at least 20% of the EVs of the present invention express N29 CAR on their surface. According to another embodiment, the N29 CAR-T cells were incubated from 12 to 36 hours with breast cancer cells expressing HER2, wherein the EVs are isolated within 24 hours post incubation. According to some exemplary embodiments, the present invention provides isolated activated extracellular vesicles, derived from N29 CAR-T cells incubated from 12 to 36 hours with ovarian cancer cells expressing HER2, wherein the EVs are isolated within 24 hours post incubation. For example, T cells may be incubated at a ratio of T cells to target cells of 1.5:1 to 3:1, e.g. 2:1. According to one embodiment, the T cells are incubated with target cells at a ratio of T cells to target cells of from 15:1 to 1:5, from 10:1 to 1:4 from 8:1 to 1:3 from 5:1 to 1:2 or from 3:1 to 1:1.

According to some embodiments, activation comprises activation with primary cancer cells obtained from the subject having the cancer. According to one embodiment, the primary cancer cells are obtained from the cancer tissue of a subject having the cancer.

According to some embodiments, the use comprises thawing of the EVs or of the pharmaceutical composition comprising the EVs prior to administration.

The pharmaceutical composition of the present invention may be administered by any know method.

The term “administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, intravenously, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the composition. Administering can also be performed, for example, once, a plurality of times, and/or over one or more extended periods. According to some embodiments, the composition is administered 1, 2, 3, 4, 5 or 6 times a day. According to other embodiments, the composition is administered 1, 2, 3, 4, 5 or 6 times a month. In some embodiments, the administration includes both direct administration, including self-administration, and indirect administration, including the act of prescribing a drug. For example, as used herein, a physician who instructs a patient to self-administer a drug, or to have the drug administered by another and/or who provides a patient with a prescription for a drug is administering the drug to the patient.

According to one embodiment, the pharmaceutical composition is formulated as a solution for injection. According to another embodiment, the pharmaceutical composition is systemically administered. According to some embodiments, the pharmaceutical composition is injected, e.g. intravenously or intramuscularly injected. According to one embodiment, the pharmaceutical composition is administered locally. According to some embodiments, the pharmaceutical composition is administered intratumorally. According to another embodiment, the pharmaceutical composition is administered in a proximity to tumor.

In some embodiments, the invention relates to the pharmaceutical compositions comprising the isolated activated EVs of the invention is for use in treating cancer as a sole anti-cancer agent.

According to some embodiments, the pharmaceutical comparison of the present invention is co-administered with an additional anti-cancer agent.

According to some embodiments, the anti-cancer compound is selected from chemotherapeutic agents, radioactive isotopes, toxins, cytokines such as interferons, and antagonistic agents targeting cytokines, cytokine receptors or antigens associated with tumor cells. In some embodiments, an anti-cancer agent is a chemotherapeutic. In other embodiments, the anti-cancer agent is CAR T-cells.

According to some embodiments, the co-administration of the pharmaceutical composition of the present invention and of additional anti-tumor compound or agent is performed in a regimen selected from a single combined composition, separate individual compositions administered substantially at the same time, and separate individual compositions administered under separate schedules and include treatment regimens in which the agents are not necessarily administered by the same route of administration or at the same time.

The term “co-administration” encompasses administration of a first and second agent in a substantially simultaneous manner, such as in a single dosage form, e.g., a capsule or tablet having a fixed ratio of first and second amounts, or in multiple dosage forms for each. The agents can be administered in a sequential manner in either order. When co-administration involves the separate administration of each agent, the agents are administered sufficiently close in time to have the desired effect (e.g., complex formation).

The term “sequential manner” refers to an administration of two compounds at a different times, and optionally in different modes of administration. The agents can be administered in a sequential manner in either order.

The terms “substantially simultaneous manner” refers to administration of two compounds with only a short time interval between them. In some embodiments, the time interval is in the range of from 0.01 to 60 minutes.

According to another aspect, the present invention provides a method of treating cancer in a subject in need thereof comprising administering an effective amount of isolated activated EVs of the present invention. According to one embodiment, the method comprises administering the pharmaceutical composition of the present invention. According to one embodiment, the cancer is selected from breast cancer, ovarian cancer, lung adenocarcinoma and stomach cancer. In some embodiments, the present invention provides a method of treating cancer selected from breast cancer, ovarian cancer, lung adenocarcinoma and stomach cancer by administering an effective amount of isolate activated EVs derived from activated CAR T-cells expressing N29 CAR, wherein at least 25% of the EVs have size of above 150 nm.

Each and every embodiment related to isolated activated EVs of the present invention as described in any one of the above aspects applies herein as well.

According to another embodiment, the method further comprises co-administration of an additional anti-cancer agent. According to some embodiments, the anti-cancer agent is a chemotherapeutic agent. According to other embodiment, the anti-cancer agent is a composition comprising CAR T-cells.

According to another aspect, the present invention provides use of the EVs according to the present invention for preparation of a medicament for treating cancer.

According to a further aspect, the present invention provides a method of preparation of the isolated activated extracellular vesicles of the present invention. According to some embodiments, the present invention provides a method for preparation of the isolated activated extracellular vesicles derived from activated CAR T-cells wherein at least 22% or at least 25% of the EVs have size of above 150 nm, wherein the method comprises incubating the CAR T-cells with a tumor associated antigen to which the CAR binds specifically under conditions enabling T cell stimulation, and isolating the derived activated extracellular vesicles.

According to some embodiments, the method of preparation comprises (1) incubating CAR T-cell with a tumor associated antigen to which CAR bind specifically, wherein the incubation is performed in a cell medium under conditions enabling T cell activation; (2) separating T-cell from the medium; (3) isolating the EVs from the medium by centrifugation at from 8,000 g to 30,000 for from 0.5 to 4 hours; 4) optionally washing the EVs; and 5) optionally freezing the EVs at a temperature below −60° C., thereby obtaining the EVs of the present invention, wherein at least 22% of the EVs have size above 150 nm. According to one embodiment, at least 25% of the EVs have size of more than 150 nm.

According to some embodiments, separating T-cell from the cell medium of step (2) comprises separating the cell medium from cells and large cell particles. The term “large cell particles” refer to cell particles above 1 μm, such as cell debris, organelles etc. As a result, cell medium comprising EVs of the present invention is obtained. The separation may be effected in one step or in several steps. According to some embodiments, separating T-cell from the medium comprises the following steps: step (2i) comprising centrifuging the medium with activated T-cell for 5 to 60 min at from 200 g to 600 g and separating/collecting the pellet, thereby separating the pellet from the medium, and step (2ii) comprising centrifuging cell medium obtained from step (2i) (supernatant) for from 10 to 60 min at from 1000 g to 3000 g and separating the resulted pellet from medium. According to some embodiments, the method comprises only step (2ii). Thus, according to some embodiments, step 2 comprises centrifuging cell medium obtained from previous step (step 1) for from 10 to 60 min at from 1000 g to 3000 g and separating the resulted pellet from the cell medium. Cell medium obtained after centrifugation may be denoted as supernatant.

According to some embodiments, step (2ii) comprises centrifuging for from 10 to 50 min, or for 10 to 30 min or for 10 to 20 min at from 1000 to 2000 g. According to some embodiments, step (2i) comprises centrifuging the medium for from 5 to 15 min at from 200 to 600 g or at about 400 g. According to some embodiments, step (2i) comprises centrifuging the medium for from 5 to 30 min at from 200 to 600 g or at about 400 g. According to some embodiments, separating T-cell from the medium comprises the following steps: step (2i) comprising centrifuging the medium with activated T-cell for 5 to 15 min at from 200 g to 600 g and separating/collecting the pellet, thereby separating the pellet from the medium, and step (2ii) comprising centrifuging cell medium obtained from step (2i) for from 10 to 30 min at from 1000 g to 3000 g and separating the resulted pellet from medium. According to some embodiments, separating the T-cell from the medium comprising the EVs comprises centrifuging for 5 to 15 min at about 400 g, and centrifuging the supernatant for 10 to 20 min at about 1500×g and collecting the supernatant for EVs purification.

According to some embodiments, the method comprises only step (2ii). In case step (2i) is absent, (2ii) comprises centrifuging cell medium obtained from step (1). According to other embodiments, the method may further comprises other steps at before step 3, wherein the centrifugation force is not above 5,000 g.

The steps (2i), (2ii) or their combination should be short enough to precipitate the cells and large cell particles, but not the EVs of the present invention. Thus, in some embodiments, a short centrifugation at step (2i) may be preferred.

Alternatively, separation of cell medium comprising EVs of the present invention from cells and large cell particles is a continuous process performed as known in art, e.g. by microfluidic system, hollow-fiber bioreactor technology (Whitford W. et al. www.GENengnews.com 2015), nanoscale separation array (Wunsch B H. Nature nanotechnology 2016), or magnetic nanowires (Lim J. J Nanobiotechnology. 2019).

According to some embodiments, the T-cells obtained in step (2i) or in step (2i) may be recycled, i.e. used for further preparation of EVs of the present invention. Thus, the cells collected in step (2i) or in step (2i) are incubated with a tumor associated antigen to which CAR bind specifically in a cell medium under conditions enabling T cell activation to initiate the process and then separated from the medium in step (2) as described above. The number of cycles in which CAR T-cells are used in preparation of the EVs of the present invention is limited only by the ability of the T-cells to generate EVs of the present invention having all properties as described above.

The terms “incubating” or “incubation” are used herein interchangeably and refers to a process of contacting or exposing CAR T-cells, with the desired entity, under conditions enabling T cell stimulation. According to one embodiment, the CAR T-cell are incubated with the TAA for at least 1 hour. According to another embodiment, the incubation is for at least 6, 12, 18 or 24 hours. According to another embodiment, the incubation is for from 1 to 96 hour. According to some embodiments, the incubation is for from 6 to 84, from 12 to 72, from 18 to 60, from 24 to 48 or from 30 to 32 hours. According to another embodiment, the incubation is for from 6 to 48, from 12 to 42, from 18 to 36 hours. According to another embodiment, the incubation is for from 20 to 30 hours. In other embodiments, the incubation is performed for about 24 hours.

In some embodiments, the EVs are isolated immediately following the incubation. According to some embodiments, the EVs are isolated within 30, 36, 42, 48, 60, 72, 84, 96 hours after separation of the T-cells from the medium. According to other embodiments, the EVs are isolated within 1, 2, 3, 4, 5, 6 or 7 days after separation of the T-cells from the medium. According to some embodiments, the EVs are isolated within 24 hours after separation of the T-cells from the medium. According to some embodiments, the EVs are isolated within 48 hours after separation of the T-cells from the medium. According to some embodiments, the EVs are isolated within 72 hours after separation of the T-cells from the medium.

According to other embodiment, upon purification the ratio of EVs to cells is at least 2, 3, 4, 5, 6, 8 or 10 times or alternatively and typically at least 50, at least 100, at least 500 or at least 1000 times higher than in the initial material According to some embodiments, the purification provides EVs substantially free of cells. According to other embodiment, purification provides cell-free EVs composition. According to some embodiments, incubation of CAR T-cell with a TAA comprises incubation with a complete TAA, a part of TAA to which the CAR binds specifically (epitope or epitope-comprising portion) or with an entity that expresses said TAA such as a complete cell, an EV expressing said TAA or any carrier such as liposomes expressing said TAA. According to some embodiments, incubation comprises incubation with cells presenting the TAA.

According to some embodiments, isolating the EVs (step 3) comprises low force centrifugation of the medium comprising the EVs. According to some embodiments, the isolation comprises centrifugation at from 8,000 g to 30,000 for from 0.5 to 4 hours. According to one embodiment, the isolation comprises centrifugation at from 8,000 g to 30,000 for from 0.5 to 3 hours. According to another embodiment, the isolation comprises centrifugation at from 8,000 g to 30,000 for from 0.5 to 2 hours. According to yet embodiment, the isolation comprises centrifugation at from 8,000 g to 30,000 for from 0.5 to 1.5 hours.

According to some embodiments, the isolation comprises centrifugation at from 8,000 g to 20,000 for from 0.5 to 4 hours. According to one embodiment, the isolation comprises centrifugation at from 8,000 g to 20,000 for from 0.5 to 3 hours. According to one embodiment, the isolation comprises centrifugation at from 8,000 g to 20,000 for from 0.5 to 2.5 hours. According to one embodiment, the isolation comprises centrifugation at from 8,000 g to 20,000 for from 0.5 to 2 hours. According to one embodiment, the isolation comprises centrifugation at from 8,000 g to 20,000 for from 0.5 to 1.5 hours. According to some embodiments, the isolation comprises centrifugation at from 16,000 g to 22,000 for from 0.5 to 4 hours or from 0.5 to 3 hours or from 0.5 to 2 hours or for from 0.5 to 1.5 hours. According to some embodiments, the isolation comprises centrifugation at about 20,000 g for 0.5 to 2.5 hours.

According to some embodiments, the isolation comprises centrifugation at from 8,000 g to 15,000 for from 0.5 to 4 hours or from 0.5 to 3 hours or from 0.5 to 2.5 hours or from 0.5 to 2 hours or for from 0.5 to 1.5 hours. According to some embodiments, the isolation comprises centrifugation at from 8,000 g to 12,000 for from 0.5 to 4 hours. According to one embodiment, the isolation comprises centrifugation at from 8,000 g to 12,000 for from 0.5 to 3 hours. According to one embodiment, the isolation comprises centrifugation at from 8,000 g to 12,000 for from 0.5 to 2 hours. According to one embodiment, the isolation comprises centrifugation at from 8,000 g to 12,000 for from 0.5 to 1.5 hours. According to another embodiment, the isolation comprises centrifugation at from 8,000 g to 12,000 for from 1 to 3 hours. According to yet another embodiment, the isolation comprises centrifugation at from 8,000 g to 12,000 for from 2 to 4 hours. According to some embodiments, the isolation comprises centrifugation at from 8,000 g to 10,000 for from 0.5 to 4 hours or from 0.5 to 3 hours or from 0.5 to 2 hours or for from 0.5 to 1.5 hours. According to some embodiments, the isolation comprises centrifugation at from 8,000 g to 10,000 for from 1 to 4 hours or from 1 to 3 hours or from 1 to 2 hours or for from 1 to 1.5 hours. According to other embodiments, the isolation comprises centrifugation at from 8,000 g to 10,000 for from 1 to 4 hours or from 1 to 3 hours or from 1 to 2 hours or for from 2 to 4 hours.

According to some embodiments, the isolation comprises low force centrifugation. According to some embodiments, the isolation comprises centrifugation at from 15,000 g to 25,000 for from 0.5 to 4 hours. According to one embodiment, the isolation comprises centrifugation at from 15,000 g to 25,000 for from 0.5 to 3 hours. According to another embodiment, the isolation comprises centrifugation at from 15,000 g to 25,000 for from 0.5 to 2.5 hours. According to another embodiment, the isolation comprises centrifugation at from 15,000 g to 25,000 for from 0.5 to 2 hours. According to yet embodiment, the isolation comprises centrifugation at about 20,000 for from 0.5 to 1.5 hours.

According to some embodiments, the CAR is anti-HER2 CAR. According to some embodiments, the CAR in N29 CAR. According to certain embodiments, activation comprises activation of anti-HER2 CAR T cells, such as N29 CAR T-cell with ovarian cancer cells presenting HER2, such as SKOV cells. According to some embodiments, activation comprises activation of anti-HER2 CAR T cells, such as N29 CAR T-cell with breast cancer cells presenting HER2. According to some embodiments, the incubation is for from 9 to 48 hours. According to some embodiments, the incubation is for from 12 to 36 hours. According to some embodiments, the incubation is for from 18 to 36 hours. In some embodiments, the derived activated extracellular vesicles are isolated immediately or within 24 or within 48 hours after separation of the CAR T cells from the medium.

According to some embodiments, the method comprises washing the EVs pellet at least once, e.g. 1, 2, 3 or more times at step (4).

According to some embodiments, the method further comprises freezing the EVs at a temperature below −60° C., e.g. at −80° C. or below. According to some embodiment, a cryoprotecting buffer may be used to protect the EVs during the freezing process.

According to some embodiments, the method for preparation is Method 1 a described in the Examples. According to some embodiments, the method for preparation is Method 5 a described in the Examples. According to some embodiments, the method for preparation is Method 3 as described in the Examples.

Typically, the T cells comprise a CD8⁺ T cell population. According to some embodiments, the T-cells are CD8⁺ T-cells. According to other embodiments, the T-cells are CD4⁺ T-cells. According to yet another embodiment, the CAR T-cells are a combination of at least CD4⁺ and CD8⁺ CAR T-cells.

According to some embodiments, the present invention provides isolated activated extracellular vesicles prepared by the method according to any one of the above embodiments. Thus, the present invention provides isolated activated extracellular vesicles prepared by the following steps: (1) incubating CAR T-cell with tumor associated antigen to which CAR bind specifically under conditions enabling T cell stimulation, preferably for from 6 to 96 hours; (2) separating the T-cell from the medium comprising the EVs by centrifuging for 10 to 30 min at from 200 to 600 g and collecting the pellet; and centrifuging the remained medium for from 10 to 30 min at from 1000 to 3000 g and discarding/collecting the resulted pellet thereby obtaining medium comprising EVs; (4) isolating the EVs from the medium by centrifugation at from 8,000 g to 30,000 for from 0.5 to 4 hours; (5) optionally washing the EVs; and (6) optionally freezing the EVs at a temperature below −60° C., thereby obtaining the EVs of the present invention, wherein at least 22% or at least 25% of the isolated EVs have particle size of 150 nm and more. According to some embodiments, separating the T-cell from the medium comprising the EVs comprises centrifuging for 5 to 15 min at about 400 g, and centrifuging the supernatant for 10 to 20 min at about 1500×g.

According to another embodiment, the method further comprises a step of enrichment of a population of EVs comprising the CAR. The step of enrichment of CAR-EVs may comprise use of magnetic beads conjugated to the specific antibodies against CAR or against CD3 or by any other sorting method.

The terms “comprising”, “comprise(s)”, “include(s)”, “having”, “has” and “contain(s),” are used herein interchangeably and have the meaning of “consisting at least in part of”. When interpreting each statement in this specification that includes the term “comprising”, features other than that or those prefaced by the term may also be present. Related terms such as “comprise” and “comprises” are to be interpreted in the same manner. The terms “have”, “has”, having” and “comprising” may also encompass the meaning of “consisting of” and “consisting essentially of”, and may be substituted by these terms. The term “consisting of” excludes any component, step or procedure not specifically delineated or listed. The term “consisting essentially of” means that the composition or component may include additional ingredients, but only if the additional ingredients do not materially alter the basic and novel characteristics of the claimed compositions or methods.

As used herein, the term “about”, when referring to a measurable value such as an amount, a temporal duration, and the like, is meant to encompass variations of +/−10%, or +/−5%, +/−1%, or even +/−0.1% from the specified value.

Having now generally described the invention, the same will be more readily understood through reference to the following examples, which are provided by way of illustration and are not intended to be limiting of the present invention.

### EXAMPLES

### Materials and Methods

### T-Cell Preparation

Peripheral human blood lymphocytes (PBL) were isolated from the blood of healthy human donors by density gradient centrifugation on Ficoll-Paque (Axis-shield, Oslo, Norway). PBLs were activated in non-tissue culture-treated 6-well plates, pre-coated with both purified anti-human CD3 and purified anti-human CD28 for 48 hours at 37° C. Activated lymphocytes were harvested and subjected to two consecutive retroviral transductions in RetroNectin pre-coated, non-tissue culture-treated 6-well plates supplemented with human IL-2 (100 IU/mL). After transduction, cells were cultured in the presence of 350 IU/mL IL-2 for 24-72 hours. Transduction efficiency was monitored by flow cytometry. Activated but non-transduced (non-infected) cells were included as T cell controls (Eshhar Z, J Immunol Methods 2001, 248:67-76).

N29 CAR targets HER2 expressed on ovarian cancer cells and anti-CD19 CAR targets CD19 expressed on hematopoietic malignant cells.

N29 CAR has a light chain variable fragment as set forth in SEQ ID NO:2 and a heavy chain variable fragment as set forth in SEQ ID NO:2. The complete CAR N29 in encoded by DNA sequence as set forth in SEQ ID NO: 4 and has amino acid sequence as set forth in SEQ ID NO: 3.

### Extracellular Vesicles Samples

Throughout the below experiments, unless stated otherwise, extracellular vesicles were obtained from N29 CAR T-cells or from Non-transduced T cells each either incubated with target cells: SKOV (HER2+) or OVCAR (HER2-), or not. The cells were incubated for 24 hours. EVs were isolated from cells medium at the end of 24 hours of cell stimulation on target cells.

The following notification of the samples is used in the Examples:

Sample 1. T cells expressing N29 CAR after stimulated with SKOV (HER2+) cells are denoted as: N29 on SKOV;
Sample 2. T cells expressing N29 CAR after incubation with OVCAR (HER2-) cells are denoted as: N29 on OVCAR;
Sample 3. Non-transduced T cells incubated with SKOV (HER2+) cells are denoted as: UT on SKOV;
Sample 4. Non-transduced T cells incubated with OVCAR (HER2−) cells are denoted as: UT on OVCAR;
Sample 5. Medium of N29 CART cells is denoted as: N29 on medium;
Sample 6. Medium of non-transduced T cells (without stimulation) is denoted as: UT on medium;
Sample 7. Target cell medium obtained from SKOV is denoted as: SKOV on medium; and
Sample 8. Target cell medium obtained from OVCAR cells is denoted as: OVCAR on medium.
In Example 5 below N29 CAR T-cells and anti-CD19 CAR T-cells were stimulated (activated) with SKOV, or incubated with Raji for 24 hours.
EVs from each of the above samples were isolated/purified by one of Methods 1-5 described below.

### Extracellular Vesicle (EVs) Isolation and Analysis

### Method 1

The medium of CAR T-cells was collected and centrifuged for 10 min at 400 g, supernatant was further centrifuged for 15 min at 1500×g. Supernatant was further centrifuged for 1 h at 20,000×g (20K×g). An EV pellet was then frozen in aliquots at −80° C. The remained supernatant was further processed according to Method 2 (below).

### Method 2

The supernatant obtained in Method 1 (after centrifugation at 20,000×g) was further centrifuged for 60 min at 100,000×g and the pellet (20K-100K×g pellet) was then frozen in aliquots at −80° C.

### Method 3

The medium of CAR T-cells was collected and centrifuged for 5 min at 400 g, supernatant was further centrifuged for 15 min at 1500×g, then supernatant was further centrifuged for 30 min at 10,000×g. The EV pellet was then frozen in aliquots at −80° C.

### Method 4

The supernatant from Method 4 was further centrifuged for 110 min at 70,000×g and the pellet was then frozen in aliquots at −80° C.

### Method 5

The medium of CAR T-cells was collected and centrifuged for 5 min at 400 g, supernatant was further centrifuged for 15 min at 1500×g, then supernatant was further centrifuged for 180 min at 10,000×g, and the pellet was then frozen in aliquots at −80° C.

### Size Assessment

EVs size and concentration were evaluated by Nanoparticle-tracking analysis (NTA) that can measured particles in the range of 50-2000 nm. NTA was performed using a NanoSight NS300 system with a CMOS camera and 532-nm laser (Malvern Instruments. Malvern, UK), each sample was measured three times. Since the EVs are mostly spherical particles, the size refers to the diameter of the EVs.

Beads having 0.7 μm size were used to set the appropriate size gate for large EVs analysis by flow cytometry. Fluorescent labeled antibodies were used to validate the expression of specific antigens.

Protein Assay

In order to calculate the amount of protein in the EVs specimens and to use the same amount of protein in each well, EVs were measured by bicinchoninic acid (BCA) a colorimetric method for detection and quantitation of total proteins or by Thermo Scientific™ NanoDrop™.

### Cells Viability—XTT Assay

In order to assess the quantity of the exposed cells and their viability, the metabolic activity of the targeted cells were analyzed. The ability of CAR T cells-EVs to reduce the tetrazolium salt XTT to orange colored compounds of formazan was measured. The intensity of the dye is correlated to the number of viable cells and was monitored by ELISA reader.

### Cell Lines

MDA231 HER2 positive breast cancer cells ( ), ovarian cancer cells, (SKOV and SKOV/Luc) (the latter stably expresses the firefly luciferase gene) and pancreatic adenocarcinoma (CAPAN) are all HER2 positive cell lines and thus are a potential target for N29 CART EVs.

MDA231 HER2 negative, Ovarian cancer cells (OVCAR cells), Raji cells, B lymphocytes of Burkitt's lymphoma, which are all HER2 negative cell and are therefore non-target cells for the EVs from N29 CAR T-cell, served as control cells.

### Cytotoxic Effect of EVs

The cytotoxic effects EVs on target cells were viewed and documented by light microscope and measured by CytoTox 96® Non-Radioactive Cytotoxicity Assay (Promega).

At the end of exposure to EVs, target cells nuclei were stained with Hoechst 33342 staining solution (ABCAM), indicating total cells number while cells apoptosis were measured by ANNEXIN/PI kit (MEBCYTO, MBL, MA, USA) according to the manufacture instruction. The cytotoxic effects of EVs on target cells were followed and documented by:

1. Light microscope (ZOE™ Fluorescent Cell Imager, Bio-Rad), analyzed by Image J software;
2. INCUCYTE (Sartorius, Germany), a live-cell imaging and analysis. The percentage of apoptotic target cells were calculated from the number of cells labeled with PI/total cells nuclei number or PI/total cells area. In addition number of cells labeled with CASPAS 3/7 or with cytotoxic dye was measured.

### Statistical Analysis

GraphPad Prism 4, Bonferroni's Multiple Comparison, one way ANOVA test and non-parametric Mann Whitney t-test.

### Example 1. Characterization of the Extracellular Vesicles Obtained in Method 1

Extracellular vesicles were obtained from T cells expressing N29 CAR or from un-transduced cells. The representative results of several measurements (n=4) of 3 different CAR T cell derived EVs.

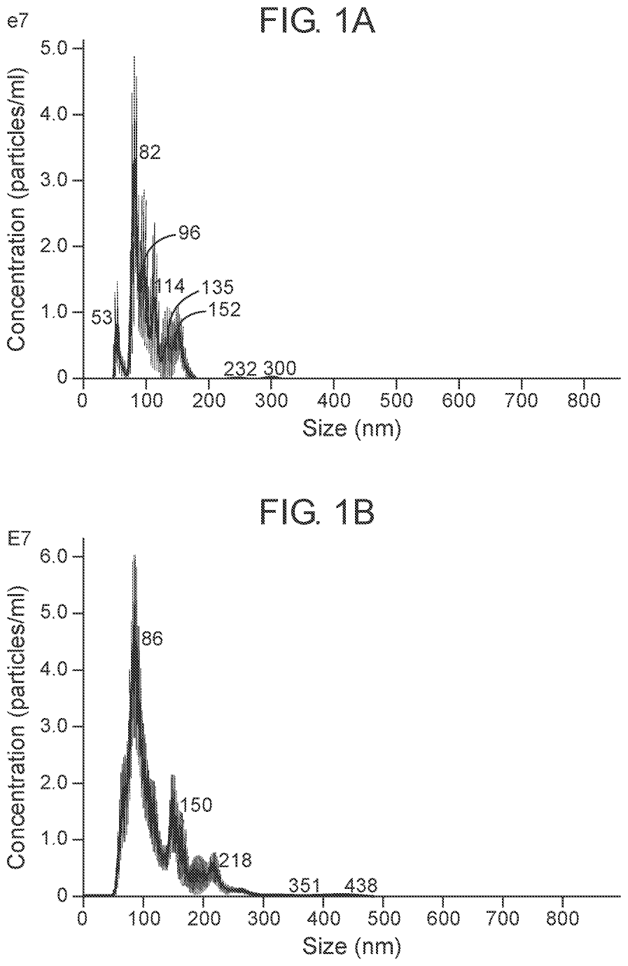

The size (the diameter) of the extracellular vesicles secreted from N29 CAR T-cells and non-transduced T cell, both incubated with HER2 expressing breast cancer cell were measured by NTA analysis and compared. N29 CAR T stimulated by target cells (HER2 positive breast cancer cells) secreted more EVs (˜25 fold) than non-transduced EVs incubated with HER2+ breast cancer cells and their EVs showed different size distribution (FIG. 1).

Additionally, it was shown that 9.54×10^{7±1.05}×10⁷ particles/ml were isolated from non-transduced T cells (10⁶/ml) after incubated with SKOV cells (0.5×10⁶/ml). The ratio of cell to EVs was found to be 1:63. In contrast total of 2.24×10⁹±1.40×10⁸ particles/ml were isolated from N29 CAR T (10⁶/ml) after stimulated by target SKOV cells (0.5×10⁶/ml). The ratio for cell to EVs found to be 1:1490.

### Example 2: Characterization of the Extracellular Vesicles Isolated by 5 Methods

EVs were obtained from 8 different samples as defined above (EVs from N29 CAR T-cell or from non-transduced cells each either incubated with SKOV or OVCAR or not), isolated by 5 different centrifugations protocols (Methods 1-5 as described above in M&M section) and characterized. The results are presented in FIG. 1 and Tables 1 and 2.

Size distribution of the EVs is presented in FIG. 1C, FIG. 1D and in Table 1. EVs were found to be significantly bigger in 20K×g pellet (obtained by Method 1) than those obtained by Method 2 (20K-100K×g pellet). This is especially correct for EVs secreted by N29 CAR T stimulated by specific target cells (HER2+SKOV cells; p value<0.01) (which also secreted more EVs, data not shown) and N29 CAR T incubated with non-specific cells (HER2-OVAR, p value<0.01) (FIG. 1D). Similarly, EVs obtained by Method 3 were significantly larger than EVs obtained by Method 4. In both case, the difference is statistically significant (p value<0.05). In fact, the EVs obtained by Methods 1 and 3 had a statistically larger particle size (p<0.001) compared to other methods mainly in CAR T EVs stimulated with target HER2+ cells. No statistically significance in the sizes of EVs obtained by Methods 5 was detected.

TABLE 1
Size of EVs obtained from Sample 1 and 2
by five different isolation methods

| | | | | | | One way |
|---|---|---|---|---|---|---|
| EVs | | | | | | ANOVA |
| size (nm) | | | | | | test between |
| (Mean, Std. | Method | Method | Method | Method | Method | methods |
| Deviation) | 1 | 2 | 3 | 4 | 5 | (M) |
| EVs N29 | 172.7 ± | 124.0 ± | 164.9 ± | 122.6 ± | 141.0 ± | M1 vs. M2 |
| on | 24.34 | 16.29 | 17.01 | 8.461 | 4.002 | p < 0.01 |
| SKOV | | | | | | M1 vs. M4 |
| Her2+ | | | | | | p < 0.01 |
| EVs size | | | | | | M2 vs. M3 |
| (nm) | | | | | | p < 0.05 |
| | | | | | | M3 VS M4 |
| | | | | | | p < 0.05 |
| EVs N29 | 167.5 ± | 121.5 ± | 141.1 ± | 130.5 ± | 142.7 ± | M1 vs. M2 |
| on | 29.07 | 18.92 | 17.17 | 12.93 | 9.779 | p < 0.01 |
| OVCAR | | | | | | M1 vs. M4 |
| Her2− | | | | | | p < 0.01 |
| EVs size | | | | | | M2 vs. M3 |
| (nm) | | | | | | p < 0.05 |

Moreover, the percent of EVs having size above 150 nm is significantly higher in 20K×g pellet (obtained by Method 1) than in pellet obtained by Method 2 for both Samples 1 and 2 (p value<0.05) (FIG. 1E, 1F and Table 2). Comparison between isolation methods demonstrated that more than 40% of the EVs obtained in pellet of Method 1 from N29 on SKOV sample had size above 150 nm, whereas the pellet of Method 2 (20K-100K×g) comprised only about 20% of such EVs. The pellet of EVs obtained from Sample 1 by Method 3 (10,000×g 30 min) contained also high rate of large EVs, comparable to that seen in the pellet obtained by Method 1, and much higher than in pellet obtained by Method 4.

TABLE 2
Percent of EVs having size above 150 nm

| % EVs | | | | | | One way |
|---|---|---|---|---|---|---|
| larger than | | | | | | ANOVA |
| 150 nm | | | | | | test between |
| (Mean, Std. | Method | Method | Method | Method | Method | methods |
| Deviation) | 1 | 2 | 3 | 4 | 5 | (M) |
| EVs N29 | 41.57% | 20.52% | 60.91% | 19.50% | 26.32% | M1 vs. M2 |
| on | ± | ± | ± | ± | ± | P < 0.05 |
| SKOV | 11.18 | 6.24 | 12.43 | 3.75 | 13.22 | M1 vs. M4 |
| Her2+ | | | | | | P < 0.01 |
| | | | | | | M2 vs. M3 |
| | | | | | | P < 0.001 |
| | | | | | | M3 vs. M4 |
| | | | | | | P < 0.001 |
| | | | | | | M3 vs. M5 |
| | | | | | | P < 0.001 |
| EVs N29 | 39.36% | 22.99% | 29.41 | 22.66 | 28.60 | M1 vs. M2 |
| on | ± | ± | ± | ± | ± | P < 0.05 |
| OVCAR | 13.50 | 9.560 | 9.392 | 6.764 | 4.775 | M2 vs. M3 |
| Her2− | | | | | | P < 0.001 |

As can be seen from FIG. 1G, Method 5 provided higher yield of EVs having size above 150 nm. About 4.1×10¹⁰ (±3.8×10¹⁰) EVs were obtained from Sample 1 purified by Method 1 and about 3.6×10¹¹ (±1.1×10¹¹) EVs were obtained from Sample 1 purified by Method 5. Although Sample 2 provided larger amount of EVs larger than 150 nm, these EVs have no cytotoxic effect on cancer cells, as shown below, and therefore have no therapeutic value.

For large EVs (>300 nm) membrane antigen characteristics by Flow cytometry, a gate for EVs size was set using the 0.75 μm beads indicating that we measured EVs that are under 1 micron size FIG. 1. H show side scatter (SSC) versus forward scatter (FSC) plots using 0.75 μm beads. R1 gate characterized the area were these beads accumulated in the graph. FIG. 1I and FIG. 1J presented FACS analysis of EVs of Sample 1 and Sample 2 both obtained by Method 1. EVs of both samples fall within the R1-gated region set indicating that we measured EVs under 1 micron size.

### Example 3. Characterization of EVs Antigen Expression on EVs Using Isolation Methods 1 and 2

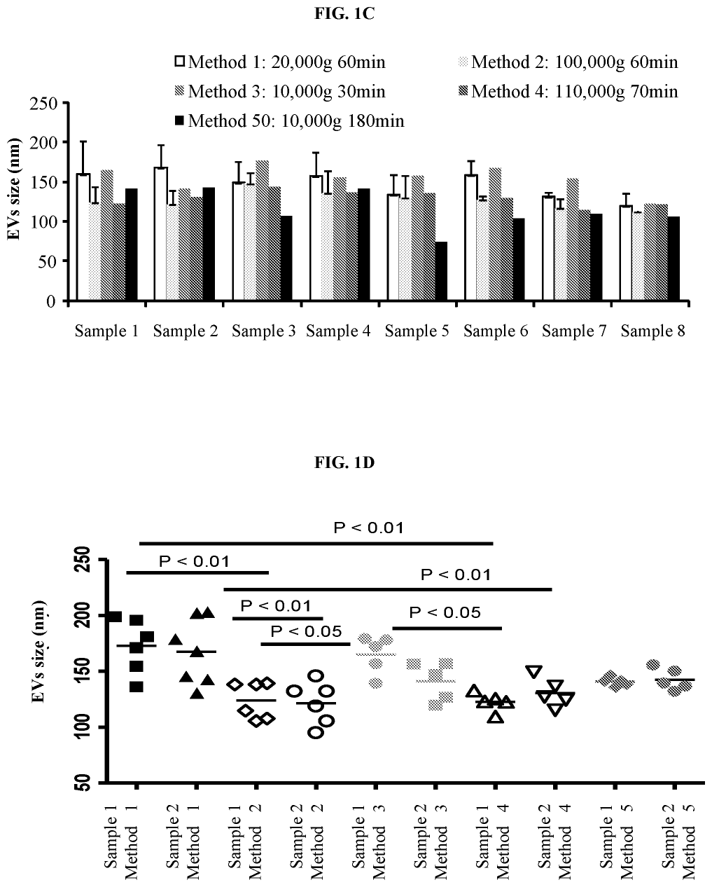

EVs were analyzed for expression of CD3 (antigen characterizing T-cells) in comparison to nonspecific labeling with isotype control IgG or to unstained EVs. It can be seen from FIGS. 2I-2L that approximately 30% of the measured EVs expressed CD3 in comparison to the control (nonspecific labeling (FIGS. 2E-2H)) or to unstained EVs (FIGS. 2A-2D).

Expression of CD3, CD38 and HLADR antigens on EVs in the pellets obtained by Method 1 or by Method 2 from 8 samples are presented in FIG. 2M. CD38 is a glycoprotein found on the surface of white blood cells and HLADR is an MHC class II cell surface receptor encoded by the human leukocyte antigen complex. Left panel of the figure refers to EVs obtained by Method 1; right panel refers to EVs obtained by Method 2. It can be seen that EVs obtained from by Method 1 (20,000 g pellet) present significantly higher levels of all three membrane antigens than EVs obtained by Method 2. Specifically, CD3 and HLADR were expressed in about 30% of EVs of 20,000 g pellet versus about 5% and 12%, respectively, in EVs obtained by Method 2 (FIG. 2M). It can be seen that about 20% of the EVs obtained by Method 1 express CD38.

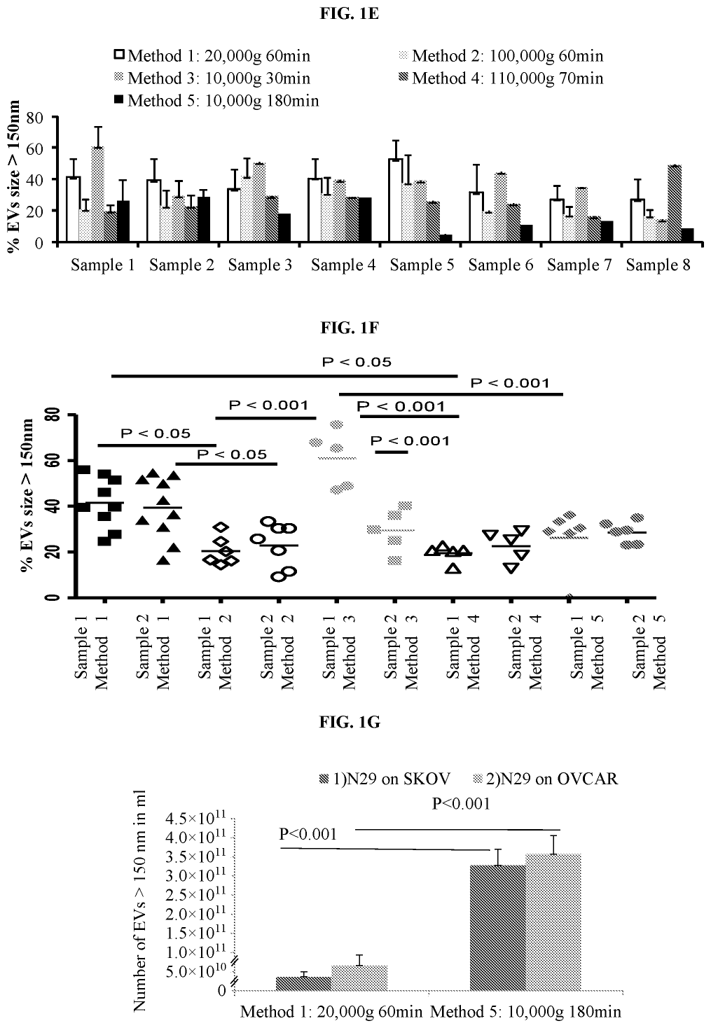

In a similar experiment, EVs derived from N29-GFP CAR T-cells or from non-transduced cells were analyzed for appearance of green color. The green color of the green fluorescent protein (GFP) in EVs was measured by florescent laser by flow cytometry. The results are presented in FIG. 3. As can be seen from that figure, about 21% of the vesicles contained GFP and were colored (FIG. 3B) in comparison with nonspecific labeling with isotype control IgG (FIG. 3A) or with EVs obtained from un-transduced T cells (FIG. 3C).

### Example 4. Cytotoxicity of EVs Obtained by Methods 1 and 2

Cytotoxicity effects of EVs from 8 Samples purified by Methods 1 or 2 on target cells were studied according to experimental arrangements as described in Table 3.

TABLE 3
Experimental arrangement for measuring EVs cytotoxicity.

| | | | | | | | | |
|---|---|---|---|---|---|---|---|---|
| EVs | 1.N29 | 2.N29 on | 3.UT | 4.UT on | 5.N29 on | 6.UT on | 7.SKOV | 8.OVCAR |
| pellet by | on | OVCAR | on | OVCAR | Medium | Medium | on | on Medium |
| Method 1 | SKOV | | SKOV | | | | Medium | |
| EVs | 1a.N29 | 2a.N29 | 3a.UT | 4a.UT on | 5a.N29 | 6a.UT on | 7a.SKOV | 8a.OVCAR |
| pellet | on | on | on | OVCAR | on | Medium | on | on Medium |
| Method 2 | SKOV | OVCAR | SKOV | | Medium | | Medium | |

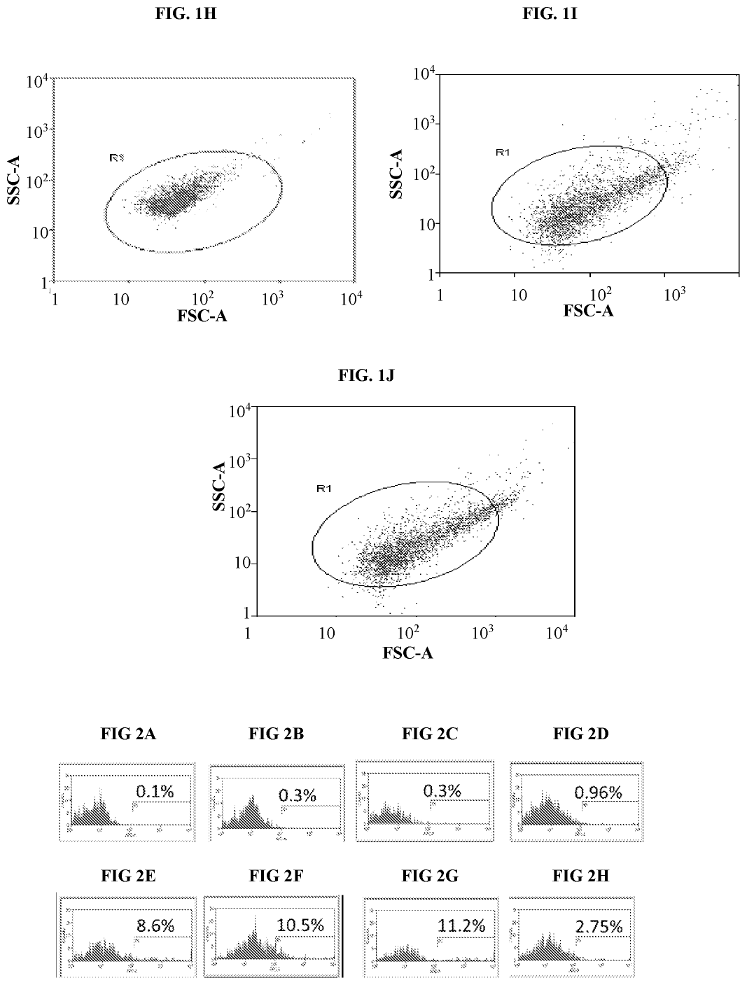

Short time exposure (6 h) of SKOV cell to two different doses (25 μg/well and 12.5 μg/well total proteins) of EVs obtained from Sample 1, (N29 CART stimulated on HER2+ cells) induced massive apoptosis of target cells as can be seen in microscopy images (FIG. 4A rows 2&3) using staining with ANNEXIN-V and PI (white dotes). Hoechst, a nucleic acid staining was used for total cells count; co-localization of ANNEXIN-V (green), PI (red) and Hoechst, (blue) documented as a merge images.

EVs obtained from Sample 2 (N29 CAR T incubated with OVCAR), did not affect SKOV cells viability (FIG. 4A 4^{th} row). Furthermore, OVCAR (HER2-) cells viability was not affected by any of the EVs populations in short time exposure (6 h) FIG. 4B.

FIG. 4C summarizes the effects of EVs from Samples 1 and 2 isolated by Method 1 and by Method 2 that were used fresh (within 24 hours after isolation) or after storage in −80° C. The percentage of apoptotic effect induced by these EVs on SKOV HER2+ cells presented in the left panel and on OVCAR HER2-cells presented in the right panel. Only EVs purified by Method 1 from Sample 1 (N29 on SKOV (HER2+)) either fresh (p value<0.001) or frozen and thawed (p value<0.05), induced statistically significant apoptosis of SKOV cells in comparison to all other EVs populations. In average, about 60% of cells were killed after exposure of 6 h EVs from Sample 1 purified by Method 1. EVs obtained from Sample 1 and purified by Method 2 and EVs obtained from Sample 2 (N29 CAR T cell incubated with non-target cells OVCAR (regardless of the purification method)) showed low toxicity and were comparable to control (no EVs). No significant apoptotic effect to OVCAR (HER2 negative cells) was observed for any type of the tested EVs. Images analysis performed by Image J software.

FIG. 4D summarize the apoptotic effect of representative different EVs populations (50 μg/well and 25 μg/well EVs) on SKOV Her2+ cells (left side) and on OVCAR Her2-cells (right side).

Only EVs obtained from Sample 1 and purified by Method 1, induced significant apoptosis rate (about 60%) compared to all other EV populations.

FIG. 4E summarize the apoptotic effect of 8 different EVs samples obtained by Method 1 and by Method 2 (50 μg/well and 25 μg/well EVs) on SKOV HER2+ cells.

### Example 5. Cytotoxicity of EVs

### Study Design

EVs were obtained from T-cells transfected with N29 or with anti-CD19 CARs. Some of the CAR T-cells were activated with SKOV or Raji cells prior to generation of EVs. The EVs were purified by Method 1 Ovarian cancer. SKOV cells were exposed to different samples comprising EVs according to Table 4.

TABLE 4
Study design

| Sample | 1 | 2 | 3 | 4 | 5 | 6 | 7 | 8 | 9 | 10 | 11 |
|---|---|---|---|---|---|---|---|---|---|---|---|
| EV derived | N29 | CD19 | UT | without | N29 | CD | UT | without | N | CD | without |
| from CAR | | | | EVs | | 19 | | EVs | 29 | 19 | EVs |
| T-cells | | | | | | | | | | | |
| transduced | | | | | | | | | | | |
| with: | | | | | | | | | | | |
| Stimulation | Skov | Skov | Skov | Skov | Raji | Raji | Raji | Raji | — | — | — |
| of CAR T- | | | | medium | | | | | | | |
| cells with: | | | | | | | | | | | |

*UT-non-transduced;

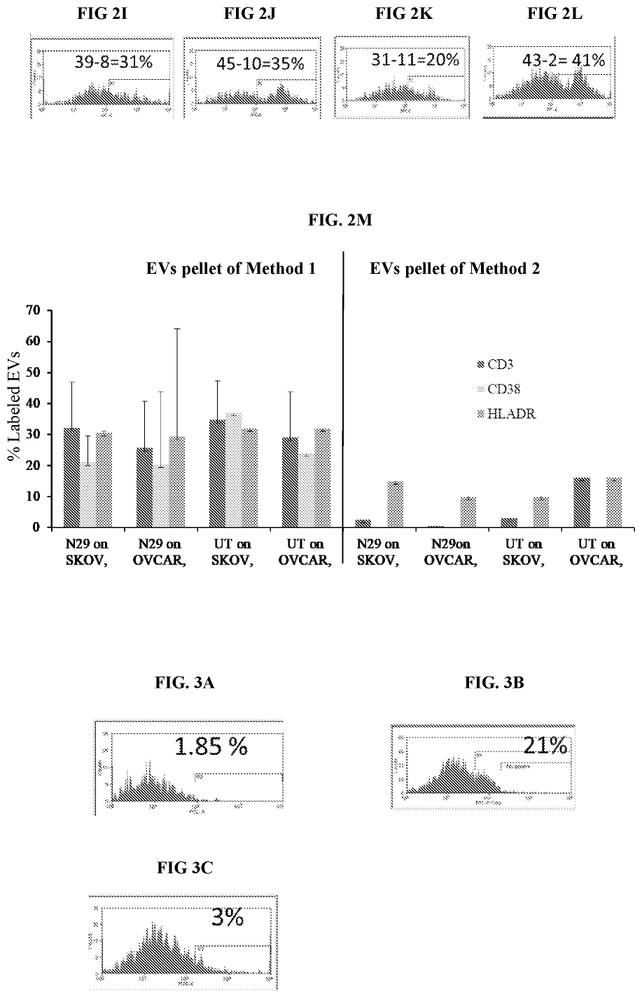

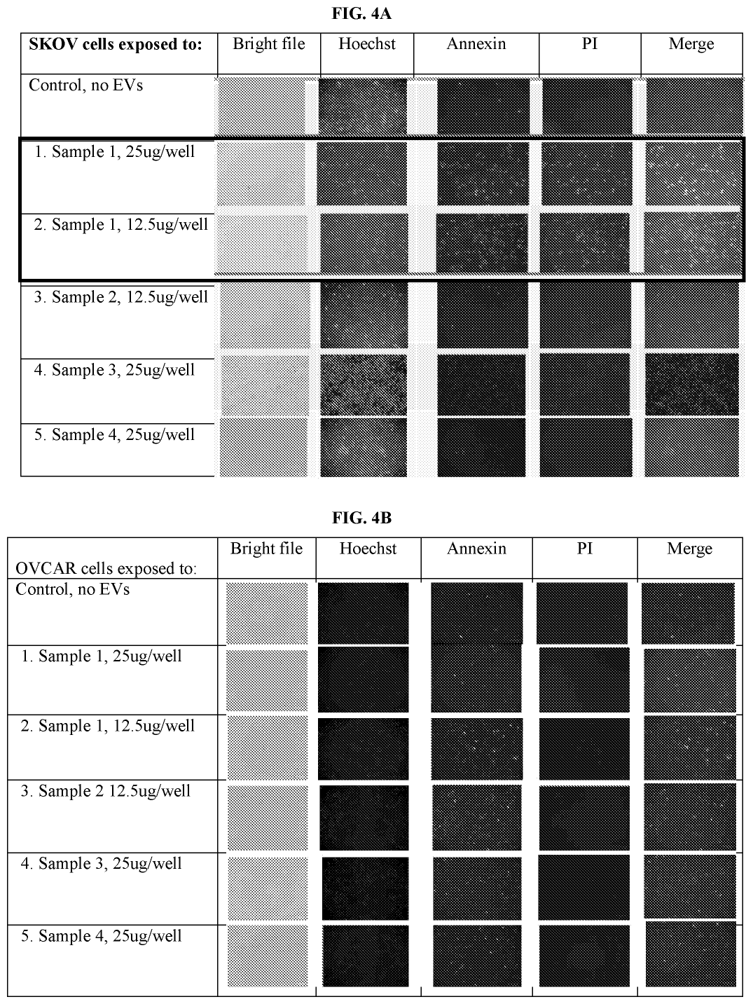

SKOV cells were seeded in 96 wells plate (10,000 cells/well) and were exposed for 48 hours to EVs population obtained from similar amount of cells. After 20 hours and 40 hours of the exposure, cells were visualized by light microscope (×10, ×20) and fluorescent microscopy and photographed. The results are presented in FIG. 5 (20 hours incubation) and 6 (40 hours incubation). Morphological changes of SKOV cells can be clearly seen in FIG. 5A and FIG. 6A showing SKOV cells exposed for 20 and 40 hours, respectively, to EVs derived from N29 CAR T-cells activated by SKOV cells. The cytotoxicity effects include morphology distraction of the cells gap junction, cell elongation and cell death. These changes were neither observed when SKOV cells were exposed to EVs derived from anti-CD19 CAR T-cells (FIGS. 5B, 5D, and 6B) nor when the EVs were derived from N29 CAR T-cells that were exposed to non-specific antigen (FIG. 5C) and nor when the EVs were derived from N29 CAR T-cells that were exposed to non-specific antigen were added to HER2 negative MDA231 cells (FIG. 6C). It can be seen that only EVs derived from N29 CAR T-cells that were stimulated with specific antigen, i.e. with cell expressing HER2, were cytotoxic to SKOV cells. On the contrary, the EVs derived from CAR T-cells stimulated with non-specific antigen showed no cytotoxic effect.

### Example 6. Cytotoxicity of EV

SKOV cells were incubated for 40 hours with EVs derived from activate GFP-N29 CAR T-cells. The overlay of the fluorescent image and the bright field image demonstrated that the EVs comprising green N29 CARs penetrated specifically into the ovarian cancer (SKOV) and colored the recipient cells in green (data not shown).

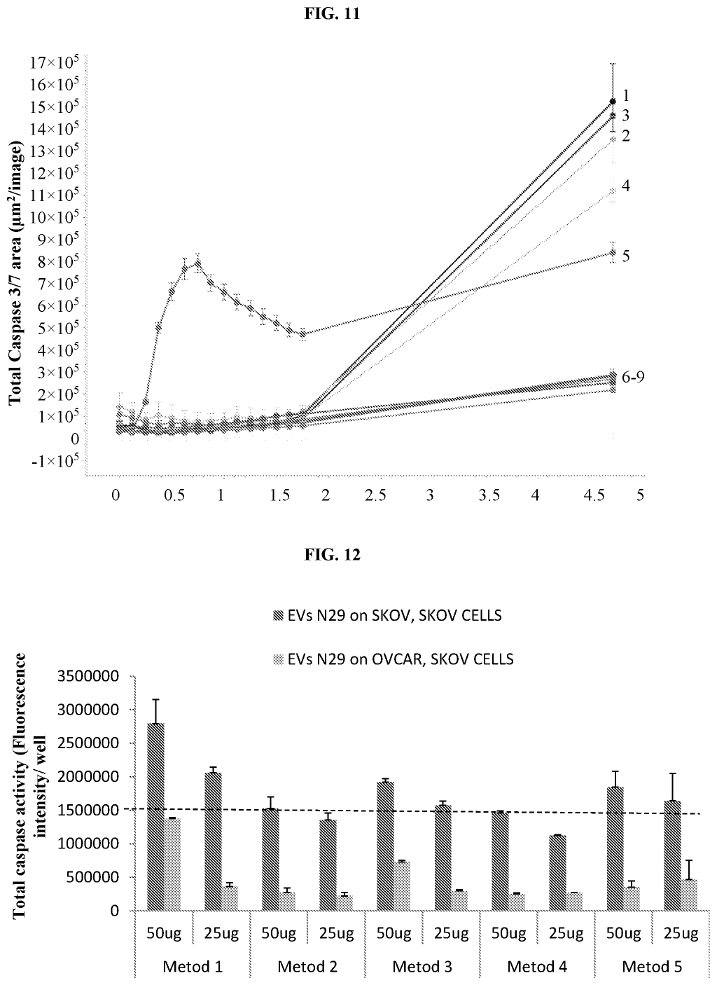

Cells viability/proliferation was measured after 48 hours by XTT method. A significant reduction in cell metabolism was found in cells exposed to EVs obtained from N29 CAR T-cells incubated with SKOVs in comparison to cells exposed to EVs obtained from un-transduced T cells (FIG. 13)

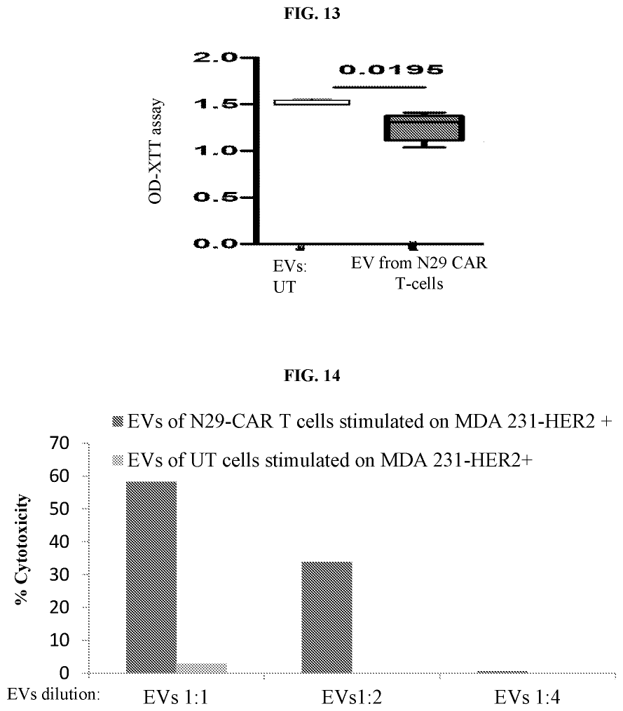

In addition, EVs cytotoxicity was measured by CytoTox 96® Assay. The assay quantitatively measures lactate dehydrogenase (LDH), a stable cytosolic enzyme that is released upon cell lysis. EVs obtained from N29 CAR T-cells activated with MDA-231—HER2 positive breast cancer cells, induced 60% and 33% killing (in a dose response manner) of MDA-231—HER2 positive breast cancer cells. In contrast, only 3% killing effect was induced by EVs obtained from UT T cells incubated with MDA-231 HER2 positive breast cancer cells (FIG. 14).

### Example 7

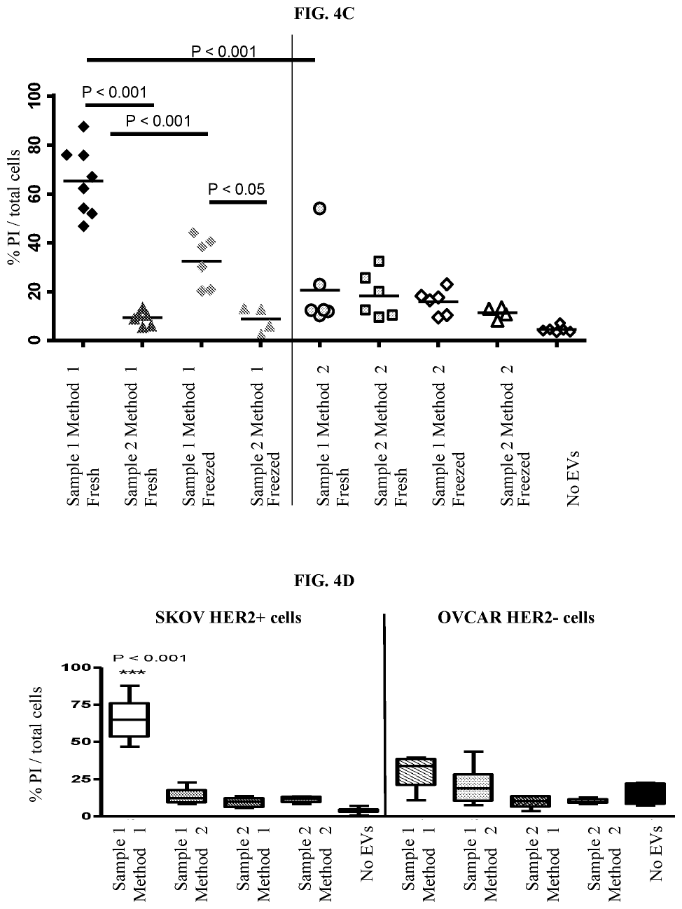

Cytotoxicity of EVs from Samples 1-4 isolated by 5 different centrifugation Methods 1-5 on SKOV cells was further tested by measurement of caspase 3/7 activity. SKOV cell were exposed to 2 different concentrations (25 or 50 μg/well) of different EVs populations and were documented every 3 hours in the first 2 days and then after 4 days. Results are present in FIG. 7. FIG. 7 shows co-localization of phase images and labeled cells with fluorescent Caspase 3/7 activity dye and with Cytotoxic Reagent (Counting Dead Cells) documented in white spots. FIG. 7A presents the effects of EVs obtained by method 1, 3 and 5. FIG. 7B presents the effects of EVs obtained by method 2 and 4.

Massive apoptosis of cells and incorporation of caspase 3/7 activity dye was found in cells exposed for 4 days to 50 μg/well, 25 m/well of EVs from Sample 1 (N29 on SKOV) obtained by Methods 1, 3 and 5 (low centrifugation force) but not in cells exposed to EVs from Sample 2 (N29 on OVCAR) that were isolated by the same methods (Methods 1, 3 and 5). EVs obtained from Sample 1 purified by Methods 2 or 4 caused only moderate rate of apoptosis. EVs obtained from Sample 2 purified by Methods 2 or 4 did not affects cells viability. Staurosporine is ATP-competitive kinase inhibitor, used to induce apoptosis, and serve as positive control for cytotoxic effect.

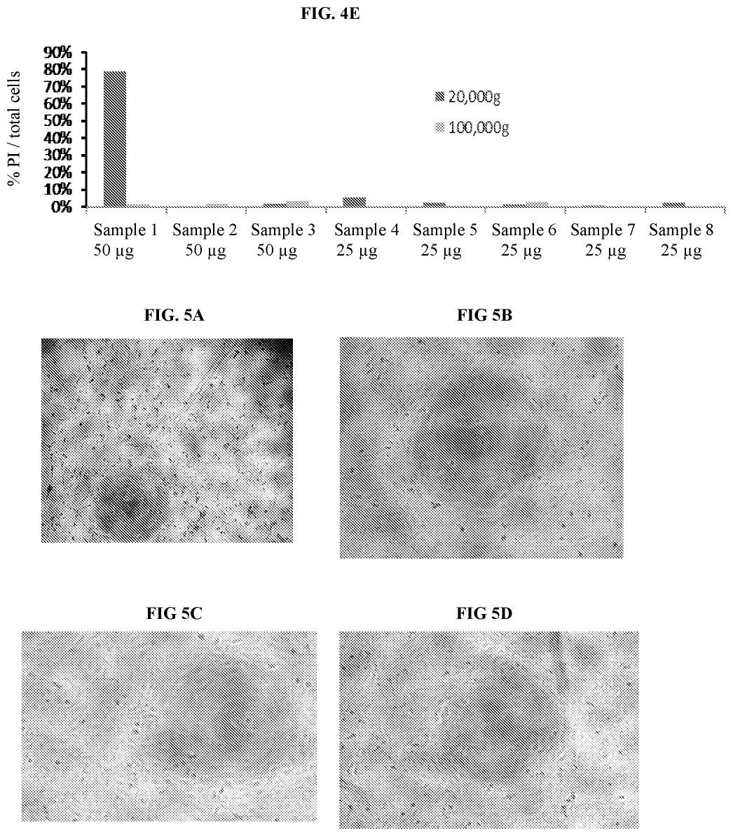

In addition, as can be seen from FIG. 8 morphologic changes were observed in SKOV cells after exposure to EVs from Sample 1 that were obtained by the 3 methods of low force centrifugation (Methods 1, 3 and 5). The cells became elongated, non-adherent apoptotic round, with big gaps between cells after exposure to the high dose (50 m/well, FIGS. 8 A, C, and E) and also after exposure to the low dose of these EVs (FIGS. 8F, H, and J).

EVs of Sample 1 that were obtained by the 2 methods of high force centrifugation (Methods 2 and 4 in both doses) induce only moderate effects on the SKOV Her2+ cells (EVs 504 well: FIGS. 8 B and D; EVs 25 m/well: FIGS. 8G and I) the apoptotic rate were similar to the rate induced by staurosporine (FIG. 8U).

Cells exposed to EVs of Sample 2 (N29 on OVCAR) isolated by the same methods did not show such morphologic changes; the cells proliferated and covered 100% of the wells area (EVs 50 m/well: FIGS. 8K-O; EVs 25 m/well: FIGS. 8 P-T).

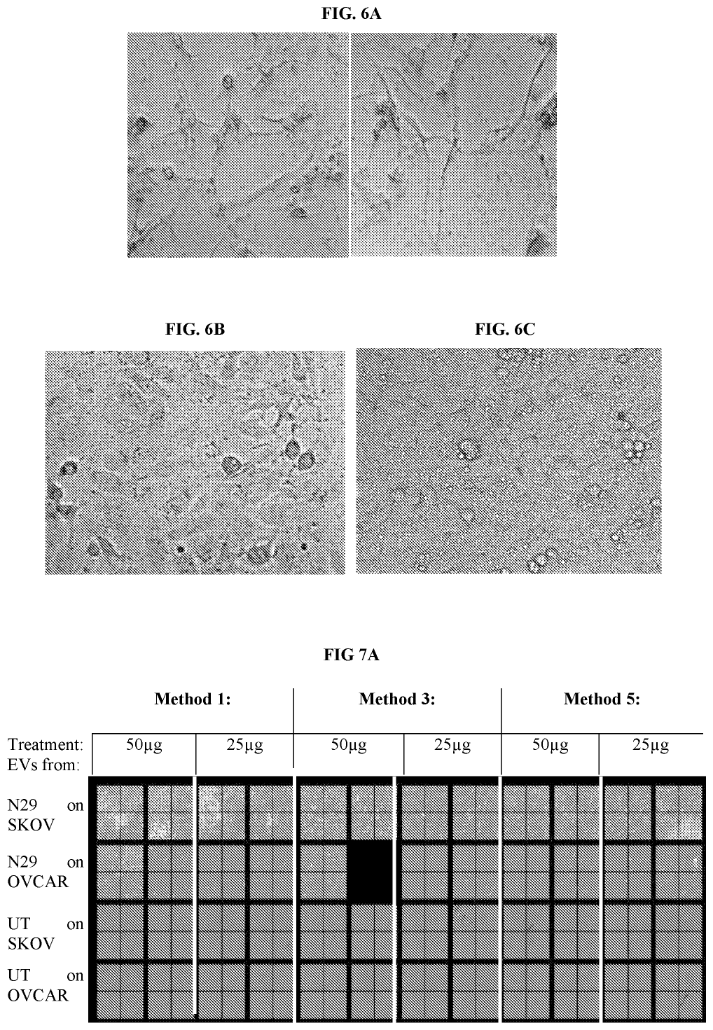

In contrasts, minimal effects were seen in OVCAR cells morphology (FIG. 9). Exposure of OVCAR HER2 negative cells to EVs from Sample 1 isolated by 5 methods induced small gaps between cells (FIGS. 9A-J) in comparison to massive apoptosis induced by staurosporine (FIG. 9U). Cell exposure to EVs of Sample 2 obtained by the 5 methods did not affect the cells and they presented massive proliferation (FIGS. 9K-T).

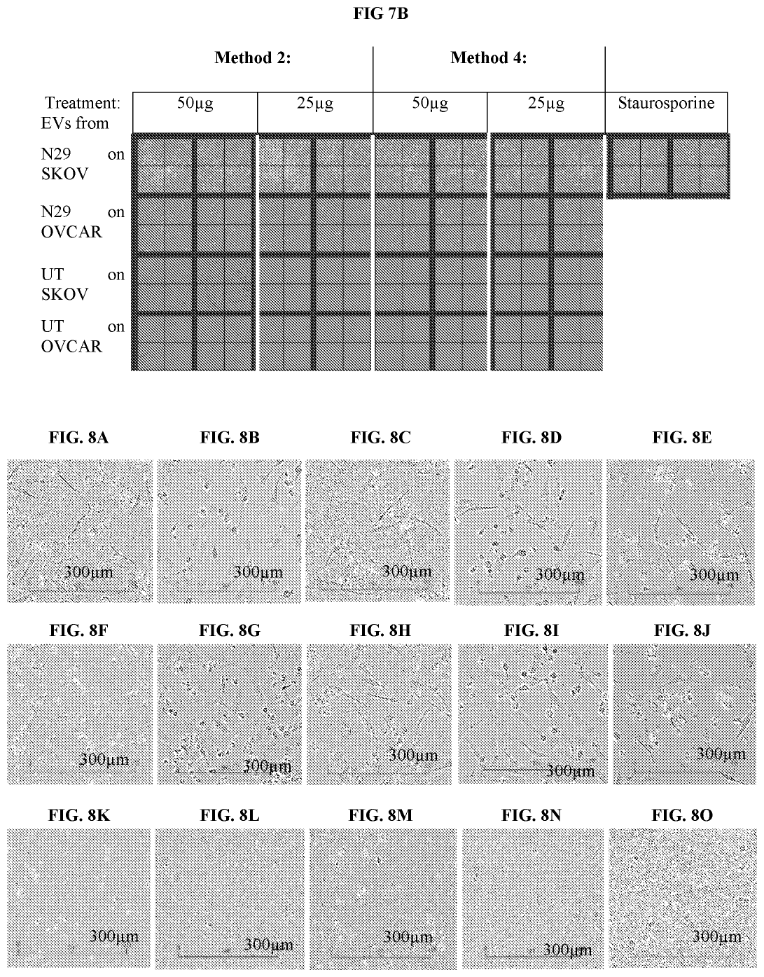

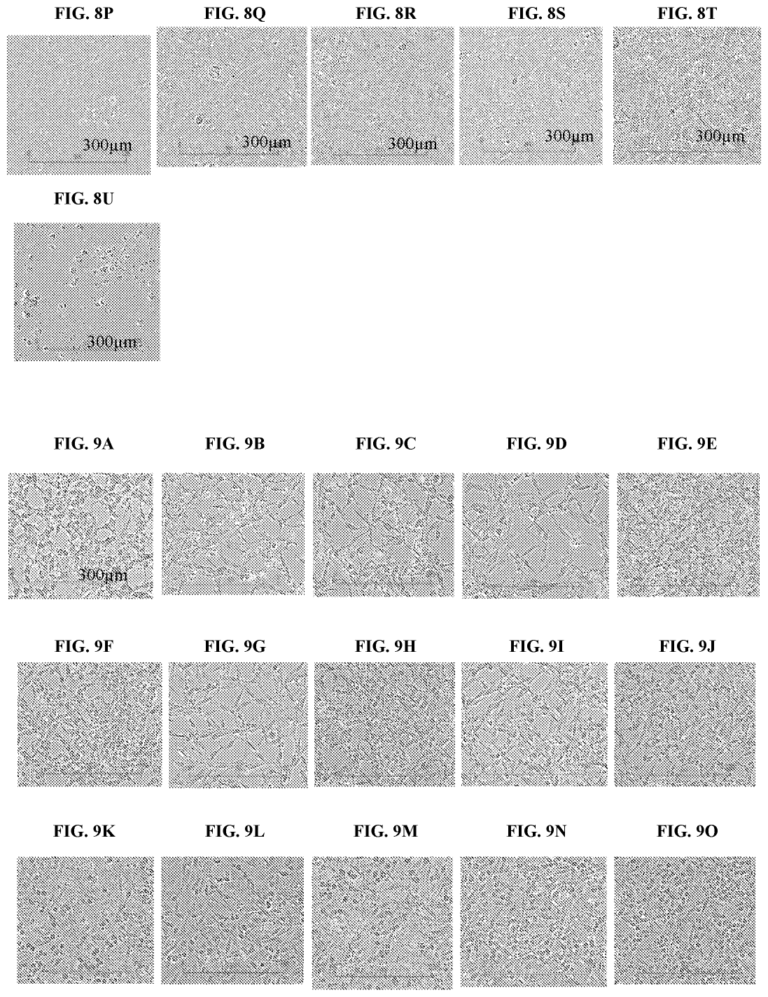

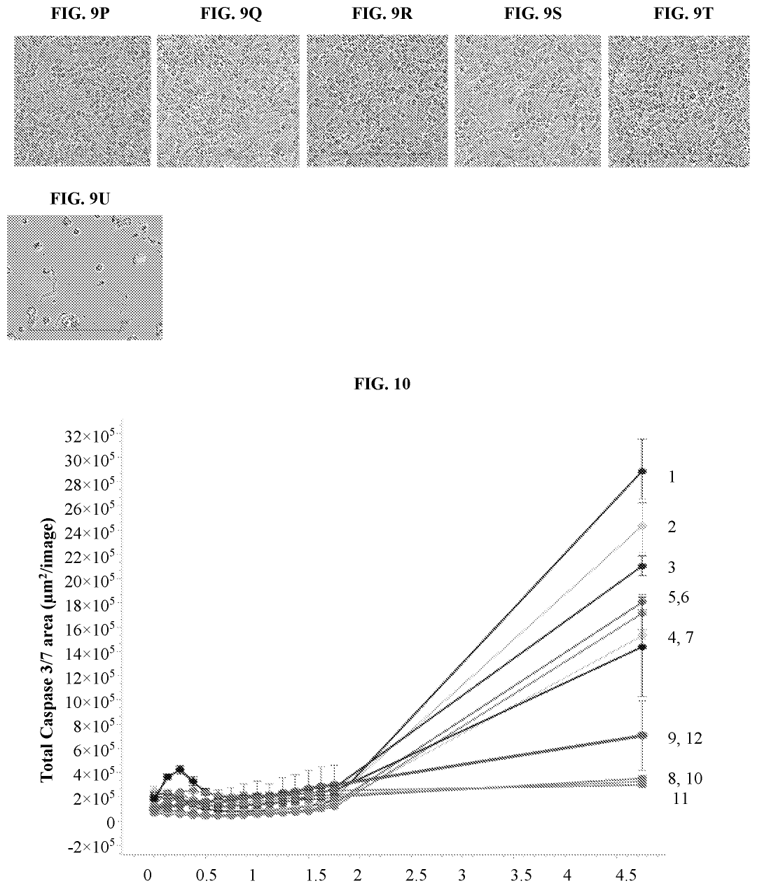

Kinetics of incorporation of fluorescent Caspase 3/7 activity marker in SKOV cells after exposure to EVs of Samples 1 and 2 obtained by Methods 1, 3 and 5 are presented in FIG. 10. It is clearly shows that the high dose (50 m/well) of EVs of Sample 1 obtained by Method 1 induced the highest Caspase 3/7 activity marker incorporation. The lower dose of these EVs (25 m/well) and both doses of EVs obtained by Method 3 and Method 5 also showed high Caspase 3/7 activity marker incorporation, which is indicative to apoptotic rate. In contrast, the high dose of EVs of Samples 2 (N29 on OVCAR) isolated by Method 1 or 3 induced moderate incorporation of Caspase 3/7 activity marker. Moreover, low concentration of EV obtained from Sample 2 by Methods 1 or 3, and any concentration of EVs from Sample 2 obtained by Methods 2 or 4 (N29 on OVCAR) did not affects the SKOV cells and did not induce Caspase 3/7 activity.

### Summary of the Results

Summarizing all the presented results it can be clearly seen that only population of EVs from Sample 1 (N29 on SKOV) and purified by Methods 1, 3 and 5 provided a strong toxic (apoptotic) effect on HER2 positive cells: such as ovarian cancer cells (SKOV) cells and HER2 positive breast cancer cells (MDA231). These populations comprise high content of large EVs, at least 25% of the EVs have size of 150 nm. The best results were obtained by EVs from Sample 1 purified by Method 1. At least 30% of these EVs have size above 150 EVs nm. It also can be seen that EVs obtained by Methods 2 and 4 that comprised much smaller EVs (in fact comprised mostly exosomes) did not generate high rate of apoptosis, and definitely not at the same level as samples with high content of large EVs.

### Example 8. Scale-Up Production

In order to increase the EVs yield we use different bioreactor systems. Hollow fiber bioreactors (HFBRs) have increasingly been implemented for EV production. In these dynamic setups, cells are expanded on cylindrical hollow fibers, which can host 100-fold more cells than common T-flasks (M. Lu, Eur. J. Pharm. Biopharm. 2017). Alternative bioreactors we use are the Quantum bioreactor culture system (Terumo BCT) (Mendt et al., JCI Insight. 2018; 3(8):e99263.2018) and the Sartorius benchtop bioreactor system.

### Example 9. CAR-T Activation

In order to facilitate the stimulation of CAR T-cells toward manufacturing “off the shelf” CAR-T cell derived activated EVs, we stimulate CAR-T cells with the antigen which is coated to the tissue culture plates. This overcomes the need of using target cells, growing them and side effects that may be caused by remnants or residual components of these target cells. Several binding protocols are tested to enhance the accessibility of antigen to the CART in order to form the immunological synapse.

### Example 10

The efficacy of the EVs of the present invention in treating cancer in vivo is tested in haematological and solid tumor models. Cancer cell lines are injected to immunodeficient mice. For solid tumor models we inject cell lines originating from ovarian cancer (SKOV, OVCAR), intraperitoneally, or subcutaneously. Alternatively we use a model of pancreas tumor by injecting pancreatic cancer cells (Capan) subcutaneously or orthotopically. Cell line that originated from breast cancer (MDA-MB-231) are injected subcutaneously. For the hematological cancer model, we inject intravenously a cell line originated from lymphoma.

EVs samples 1-8 are purified from activated CAR T cell culture by method 11 (20,000 g, 60 min) or 5 (10,000 g 180 min). EVs samples are labelled with fluorescent dye such as XenoLight DiR as described before (Ohno S. Molecular Therapy 2013). The labelled EVs (4-100 μg) are injected intravenously or intratumorally to mice bearing the transplanted tumor cells twice a week for 4 weeks. 12 and 24 hours after each injection the locations of the EVs and tumor size is monitored using an IVIS. At the end of 4 weeks brain, heart, spleen, liver, lung, kidney, small intestine, colon, and tumor tissues are harvested for pathology validation.

Although the present invention has been described herein above by way of preferred embodiments thereof, it can be modified, without departing from the spirit and nature of the subject invention as defined in the appended claims.

1-42. (canceled)

43. Isolated activated extracellular vesicles (EVs) derived from activated T-cells expressing a chimeric antigen receptor (CAR T-cells), wherein at least 25% of the EVs have a particle size diameter of above 150 nm.

44. The activated EVs according to claim 43, wherein at least 30% of the EVs have a particle size of greater than 150 nm, or wherein the mean size of the EVs is at least 140 or at least 160 nm.

45. The activated EVs according to claim 43, wherein the EVs present the chimeric antigen receptor (CAR) of the activated CAR T-cells.

46. The activated EVs according to claim 43, wherein the CAR is selected from anti-HER2, anti-CD19, and anti-CD38 CAR.

47. The activated EVs according to claim 46, characterized by at least one of:
(i) the anti-HER2 CAR is N29 CAR;
(ii) the EVs are derived from N29 CAR T-cells activated by cells expressing HER2; and
(iii) the EVs are derived from N29 CAR T-cells activated by cells expressing HER2, wherein the cells expressing HER2 are selected from ovarian cancer cells, breast cancer cells, and primary cells of HER2 positive cancer.

48. The activated EVs according to claim 43, characterized by at least one of:
(i) at least at least 10% of the EVs express CD38 antigen;
(ii) at least at least 20% of the EVs express CD3 antigen;
(iii) wherein at least at least 20% of the EVs express HLARD antigen; and
(iv) the EVs are cytotoxic EVs.

49. The activated EVs according to claim 43, further comprising an anticancer agent or devoid of an exogenous anti-cancer agent.

50. A pharmaceutical composition comprising the isolated activated EVs according to claim 43, and a pharmaceutically acceptable carrier.

51. The pharmaceutical composition according to claim 50, wherein the pharmaceutical composition:
(i) comprises the said EVs as a sole anti-cancer agent or further comprises an additional anti-cancer agent; and/or
(ii) is formulated as a formulation for injection.

52. A method for treating cancer in a subject in need thereof comprising administering to the subject an effective amount of the isolated activated EVs according to claim 43, wherein the cancer cells present an antigen to which the CAR binds specifically.

53. The method according to claim 52, wherein cancer is selected from ovarian cancer, breast cancer, lung adenocarcinoma, stomach, liver cancer, pancreatic cancer, brain cancers and a hematology malignancy.

54. The method according to claim 52, wherein:
(i) the EVs are derived from T cells comprising anti-HER2 CAR and the cancer is HER2 positive cancer;
(ii) the EVs are derived from T cells comprising anti-HER2 CAR and the cancer is HER2 positive cancer selected from ovarian and breast cancer; and/or
(iii) the EVs are derived from T cells comprising N29 CAR and the cancer is HER2 positive cancer.

55. A method for preparation of the isolated activated extracellular vesicles derived from activated CAR T-cells according to claim 43, wherein the method comprises:
(1) incubating CAR T-cells with a tumor associated antigen to which the CAR binds specifically in a cell medium under conditions enabling T cell activation;
(2) separating the CAR T-cells from the cell medium; and
(3) isolating the derived activated extracellular vesicles, thereby obtaining isolated activated EVs, wherein at least 25% of the EVs have size of 150 nm or more.

56. The method according to claim 55, wherein the method is characterized by at least one of:
(i) the isolation of the EVs at step (3) comprises low force centrifugation;
(ii) the isolation of the EVs at step (3) comprises centrifugation at from 8,000×g to 30,000×g for from 0.5 to 4 hours;
(iii) the isolation is effected by centrifugation at from 8,000×g to 15,000×g for from 0.5 to 3 hours; and
(iv) the isolation is effected by centrifugation at from 15,000×g to 25,000×g for from 0.5 to 1.5 hours.

57. The method according to claim 55, wherein the incubation at step (1) comprises incubation for from 6 to 96 hours.

58. The method according to claim 55, wherein the incubation at step (1) comprises incubating CAR T-cells with cells or surfaces presenting the tumor associated antigen to which the CAR binds specifically.

59. The method according to claim 55, characterized by at least one of:
(i) step (2) comprises step (2ii) comprising centrifuging the medium of the previous step for from 10 to 60 min at from 1000 g to 3000 g and separating the pellet from medium;
(ii) the method further comprises step (2i) before step (2ii), wherein step (2i) comprises centrifuging the medium with activated T-cells from step (1) for 5 to 60 min at from 200 g to 600 g and separating the pellet from the medium; and
(iii) the CAR T-cells are N29 CAR T-cells and the tumor associated antigen is HER2.

60. The method according to claim 55, comprising incubating N29 CAR T-cells with ovarian cancer cells presenting HER2 for from 18 to 36 hours and isolating the derived activated extracellular vesicles.

61. The method according to claim 55, wherein the method further comprises (i) washing the obtained EVs; (ii) freezing the EVs; or (iii) both (i) and (ii).

62. Isolated activated extracellular vesicles prepared by a method according to claim 55, wherein at least 25% of the isolated EVs have a particle diameter size of above 150 nm.
